SEQUENCE LISTING (48 sequences)

SEQ ID NO: 1 - DNA, 22 bp, synthetic construct
  source 1..22: mol_type = other DNA
ccagagaaggttgcccaagtat

SEQ ID NO: 2 - DNA, 22 bp, synthetic construct
  source 1..22: mol_type = other DNA
tgttgacgaactccatgggaat

SEQ ID NO: 3 - DNA, 22 bp, synthetic construct
  source 1..22: mol_type = other DNA
acactgtaccagaaggcagatg

SEQ ID NO: 4 - DNA, 22 bp, synthetic construct
  source 1..22: mol_type = other DNA
ttcaagtcatggtccccatcag

SEQ ID NO: 5 - DNA, 22 bp, synthetic construct
  source 1..22: mol_type = other DNA
agatcctggatgacacctcaga

SEQ ID NO: 6 - DNA, 22 bp, synthetic construct
  source 1..22: mol_type = other DNA
accacctgtcgaaacatctacc

SEQ ID NO: 7 - DNA, 22 bp, synthetic construct
  source 1..22: mol_type = other DNA
cagagaggtggcaaggaattct

SEQ ID NO: 8 - DNA, 22 bp, synthetic construct
  source 1..22: mol_type = other DNA
gtatgcagggtcctgatagagc

SEQ ID NO: 9 - DNA, 22 bp, synthetic construct
  source 1..22: mol_type = other DNA
gccatgcaagagttcatgatcc

SEQ ID NO: 10 - DNA, 22 bp, synthetic construct
  source 1..22: mol_type = other DNA
aagtccaggtcatacttgccag

SEQ ID NO: 11 - DNA, 22 bp, synthetic construct
  source 1..22: mol_type = other DNA
cattgatcacgaccggaatgtg

SEQ ID NO: 12 - DNA, 22 bp, synthetic construct
  source 1..22: mol_type = other DNA
gagtaaacaatgtccgcgaagg

SEQ ID NO: 13 - DNA, 22 bp, synthetic construct
  source 1..22: mol_type = other DNA
atccctttctacggcatcttgg

SEQ ID NO: 14 - DNA, 22 bp, synthetic construct
  source 1..22: mol_type = other DNA
aggtggtgctcaatctggaaat

SEQ ID NO: 15 - DNA, 22 bp, synthetic construct
  source 1..22: mol_type = other DNA
tggactactttgtggccttctc

SEQ ID NO: 16 - DNA, 22 bp, synthetic construct
  source 1..22: mol_type = other DNA
tgtgctacagctttcaccagat

SEQ ID NO: 17 - DNA, 21 bp, synthetic construct
  source 1..21: mol_type = other DNA
aggtcggtgtgaacggatttg

SEQ ID NO: 18 - DNA, 19 bp, synthetic construct
  source 1..19: mol_type = other DNA
ggggtcgttgatggcaaca

SEQ ID NO: 19 - DNA, 22 bp, synthetic construct
  source 1..22: mol_type = other DNA
tacggaaaggtctgcatcacaa

SEQ ID NO: 20 - DNA, 22 bp, synthetic construct
  source 1..22: mol_type = other DNA
tagtggaggaatcatgggaggt

SEQ ID NO: 21 - DNA, 22 bp, synthetic construct
  source 1..22: mol_type = other DNA
gaaatggggctgagaaaggaga

SEQ ID NO: 22 - DNA, 22 bp, synthetic construct
  source 1..22: mol_type = other DNA
tgtagtccaggaagtcggagat

SEQ ID NO: 23 - DNA, 22 bp, synthetic construct
  source 1..22: mol_type = other DNA
gtgtggagtggtgtgaatgttg

SEQ ID NO: 24 - DNA, 22 bp, synthetic construct
  source 1..22: mol_type = other DNA
ttagaactgcagctccttctgg

SEQ ID NO: 25 - DNA, 22 bp, synthetic construct
  source 1..22: mol_type = other DNA
agagaggatgggctcaagtact

SEQ ID NO: 26 - DNA, 22 bp, synthetic construct
  source 1..22: mol_type = other DNA
ttagcacaacctcctcttcgtc

SEQ ID NO: 27 - DNA, 22 bp, synthetic construct
  source 1..22: mol_type = other DNA
gcaggagctttgagaacaactg

SEQ ID NO: 28 - DNA, 22 bp, synthetic construct
  source 1..22: mol_type = other DNA
atgacaatgcagagttcctcgt

SEQ ID NO: 29 - DNA, 22 bp, synthetic construct
  source 1..22: mol_type = other DNA
accccttctacagcaacatcag

SEQ ID NO: 30 - DNA, 22 bp, synthetic construct
  source 1..22: mol_type = other DNA
tgagagaccctccagatgctta

SEQ ID NO: 31 - DNA, 22 bp, synthetic construct
  source 1..22: mol_type = other DNA
ttgcagacaagatccagctgat

SEQ ID NO: 32 - DNA, 22 bp, synthetic construct
  source 1..22: mol_type = other DNA
aataccccaacaggaccattcc

SEQ ID NO: 33 - DNA, 22 bp, synthetic construct
  source 1..22: mol_type = other DNA
tggacaacgcttacatggagaa

SEQ ID NO: 34 - DNA, 22 bp, synthetic construct
  source 1..22: mol_type = other DNA
tgatgttcttgcccttctctcc

SEQ ID NO: 35 - DNA, 22 bp, synthetic construct
  source 1..22: mol_type = other DNA
gcttcgtgttcaccaaggagga

SEQ ID NO: 36 - DNA, 22 bp, synthetic construct
  source 1..22: mol_type = other DNA
gtcctagaccagtgttctgagc

SEQ ID NO: 37 - DNA, 22 bp, synthetic construct
  source 1..22: mol_type = other DNA
caaagagaagggcggaaaactg

SEQ ID NO: 38 - DNA, 22 bp, synthetic construct
  source 1..22: mol_type = other DNA
ccaggatattctcccgggattg

SEQ ID NO: 39 - DNA, 22 bp, synthetic construct
  source 1..22: mol_type = other DNA
gcaagctctacacctgtctctt

SEQ ID NO: 40 - DNA, 22 bp, synthetic construct
  source 1..22: mol_type = other DNA
cagttttccgcccttctctttg

SEQ ID NO: 41 - DNA, 22 bp, synthetic construct
  source 1..22: mol_type = other DNA
agtgtatcctgttgcccttctg

SEQ ID NO: 42 - DNA, 22 bp, synthetic construct
  source 1..22: mol_type = other DNA
gaccagggcctacttcaaagaa

SEQ ID NO: 43 - DNA, 22 bp, synthetic construct
  source 1..22: mol_type = other DNA
tcagacctcggatccagtactt

SEQ ID NO: 44 - DNA, 22 bp, synthetic construct
  source 1..22: mol_type = other DNA
cttctcctccgctttctgttct

SEQ ID NO: 45 - DNA, 22 bp, synthetic construct
  source 1..22: mol_type = other DNA
ctcagaagttctccagtgccat

SEQ ID NO: 46 - DNA, 22 bp, synthetic construct
  source 1..22: mol_type = other DNA
ggtatgaaccacctccccattt

SEQ ID NO: 47 - DNA, 22 bp, synthetic construct
  source 1..22: mol_type = other DNA
agatgagctgattggccagaaa

SEQ ID NO: 48 - DNA, 22 bp, synthetic construct
  source 1..22: mol_type = other DNA
agcttctcgtgtacttcctgtg

(19) United States
(12) Patent Application Publication
(10) Pub. No.: US 2026/0227411 A1
(43) Pub. Date: Aug. 6, 2026

(54) CONJUGATED METABOLITES FOR CANCER DETECTION AND TREATMENT

(71) Applicant: VANDERBILT UNIVERSITY, Nashville, TN (US)

(72) Inventor: Eunyoung CHOI, Nashville, TN (US)

(21) Appl. No.: 19/149,949

(22) PCT Filed: Feb. 9, 2024
(86) PCT No.: PCT/US2024/015099

§ 371 Date: Jul. 22, 2025

Related U.S. Application Data
(60) Provisional application No. 63/484,312, filed on Feb. 10, 2023.

(51) Int. Cl. G01N 33/58; A61K 31/445; A61K 47/54; A61P 35/00; C07D 271/12; C07D 293/10

(52) U.S. Cl. CPC G01N 33/582; A61K 31/445; A61K 47/542; A61K 47/545; A61P 35/00; C07D 271/12; C07D 293/10

(57) ABSTRACT

Disclosed herein are compounds, research tools, and methods for detecting and treating epithelial cell dysplasia, precancer, and cancer in a cell or a subject. Also disclosed herein are compounds, research tools, and methods for inhibiting a dysplastic cell, inhibiting the transition of a metaplastic cell to a dysplastic cell, and reducing the incidence of carcinoma in a cell or a subject. In some embodiments, the compounds, research tools, and methods comprise an eicosenoic acid conjugate compound. In some embodiments, the compounds, research tools, and methods comprise a therapy comprising an SCD1 enzyme inhibitor.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “093386-9357-WO01_sequence_listing_xml_2 Feb. 2024.xml,” was created on Feb. 2, 2024, contains 48 sequences, has a file size of 42.3 Kbytes, and is incorporated by reference in its entirety into the specification.

### FEDERALLY SPONSORED RESEARCH

This invention was made with government support under grant numbers R37 CA244970 awarded by the National Institutes of Health. The government has certain rights in the invention.

### TECHNICAL FIELD

Disclosed herein are compounds, research tools, and methods for detecting and treating epithelial cell dysplasia, precancer, and cancer in a cell or a subject. Also disclosed herein are compounds, research tools, and methods for inhibiting a dysplastic cell, inhibiting the transition of a metaplastic cell to a dysplastic cell, and reducing the incidence of carcinoma in a cell or a subject. In some embodiments, the compounds, research tools, and methods comprise an eicosenoic acid conjugate compound. In some embodiments, the compounds, research tools, and methods comprise a therapy comprising an SCD1 enzyme inhibitor.

### BACKGROUND

Epithelial carcinogenesis undergoes multiple steps of carcinogenic process from precancerous metaplasia to dysplasia and adenocarcinoma, and alterations of genetic, epigenetic, and metabolic pathways are involved in the process. Also, the carcinogenesis often requires oncogenic gene activation which regulates signaling pathways, including RAS signaling pathway. GI cancers are common life-threatening cancers and occur along the digestive tract epithelium including the esophagus, stomach, and pancreas. Metaplasia in GI cancers can arise from mucosal injury and is potentially reversible. However, cell plasticity also permits the entry of metaplastic cells into the carcinogenic process to dysplasia and adenocarcinoma. In particular, it has been noted that metaplasia arises from zymogen-secreting chief cell plasticity contributes in response to severe gastric injury and Kras activation and amplification can lead to gastric carcinogenesis. Dysplasia can be defined as a condition in which cells have abnormal cellular and architectural changes and is the focal neoplastic lesion, which has the highest risk of gastric cancer development. Dysplasia may evolve into adenocarcinoma when oncogenic environments chronically persist, and therefore, it is considered a key transition stage entering adenocarcinoma development in epithelial carcinogenesis. Dysplastic stem cells, a stem cell population which is first present in the dysplastic stage and evolves to gastric adenocarcinoma, was recently identified. However, the molecular and cellular mechanisms of the precancerous metaplasia progression to dysplasia and even to adenocarcinoma still remain poorly understood.

Metabolism includes all the chemical reactions that produce energy or generate biological building blocks of cells. Metabolic reprogramming is essential for supplying energy to meet the increased demands when cells need exponential growth and proliferation, especially in cancer cells. Cells stabilize metabolic pathways to control suitable levels of critical metabolites which are linked to cell signaling pathways and epigenetics. Recent studies have reported that bioactive metabolites are important for regulation of cellular heterogeneity, tumor growth, and immune response. Fatty acid (FA) metabolism in cancer is implicated in tumor progression, metastasis potential, and drug resistance. FAs are critical in cellular homeostasis in a variety of biological processes including energy supply and storage, cell signaling, transcription control, phospholipid synthesis, and membrane fluidity. FAs are synthesized de novo or obtained from the diet and modified through metabolic pathways that include desaturation and elongation steps. In particular, FA desaturation generates building blocks of membrane lipids and protects the cells against lipotoxicity of saturated fatty acids which impair membrane fluidity. Stearoyl-CoA desaturase (SCD) is a key enzyme that generates monounsaturated fatty acids (MUFAs) which contribute to lipid homeostasis. Although it is well known that the FA metabolism supports cancer cell proliferation and progression, it remains undefined whether the FA metabolism occurs to fuel dysplastic cells or even any specific metabolic pathway can regulate dysplasia proliferation or progression.

What is needed are conjugated metabolites for cancer and precancer detection and treatment.

### SUMMARY

One embodiment described herein is a compound of formula (I):

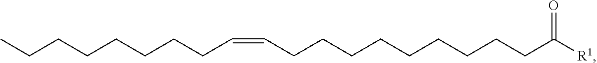

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine. In one aspect, the compound is:

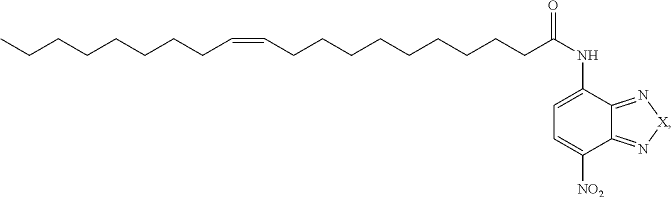

where X is O or Se.

Another embodiment described herein is a compound of formula (I):

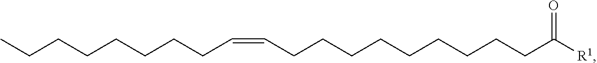

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

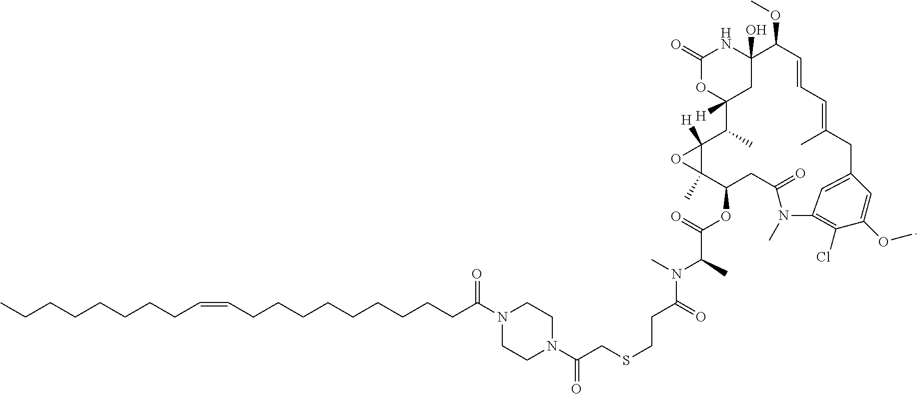 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method for detecting epithelial cell dysplasia, the method comprising: contacting an epithelial cell with a compound as described herein; incubating for a period of time; irradiating the epithelial cell with ultraviolet light, whereby a dysplastic epithelial cell will fluoresce; and imaging the fluorescent dysplastic epithelial cell. In one aspect, imaging comprises positron emission tomography (PET) imaging, mass spectrometry imaging, immunofluorescence imaging, fluorescence molecular endoscopy, or fluorescence-guided intraluminal endoscopy. In another aspect, the epithelial cell comprises a gastric epithelial cell.

Another embodiment described herein is a research tool for detecting epithelial dysplasia, the research tool comprising a compound of formula (I):

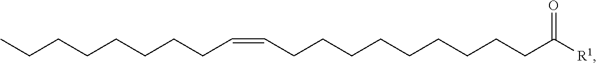

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine. In one aspect, the compound is:

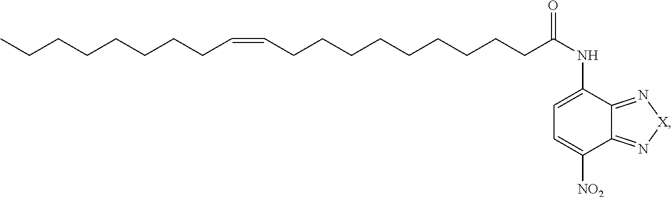

where X is O or Se.

Another embodiment described herein is a research tool comprising a compound of formula (I):

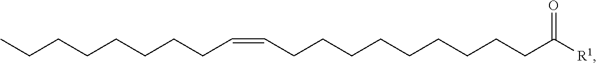

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

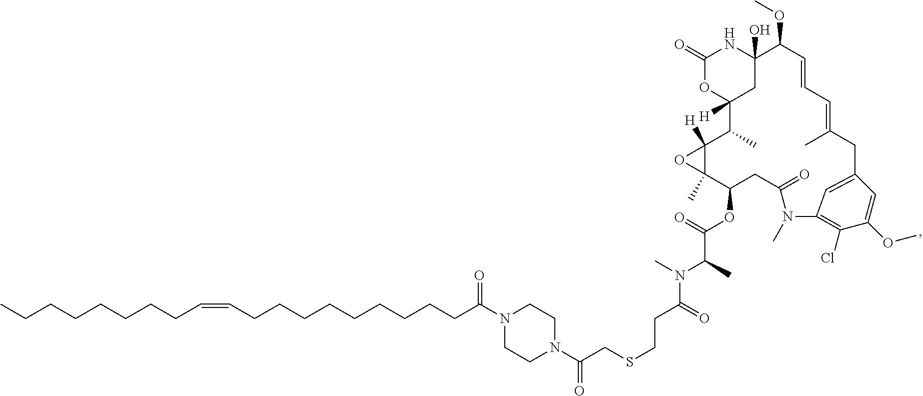 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method of inhibiting cancer or precancer in a cell or a subject in need thereof, the method comprising administering to the cell or the subject a therapeutically effective amount of a compound of formula (I):

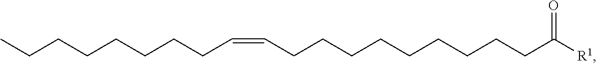

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

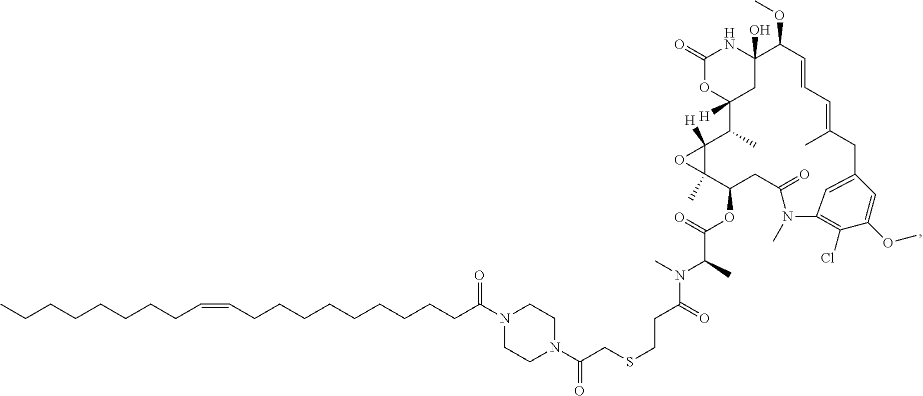 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method of treating a subject having cancer or precancer, the method comprising administering to the subject a therapeutically effective amount of a compound of formula (I):

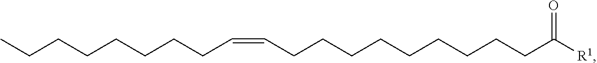

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

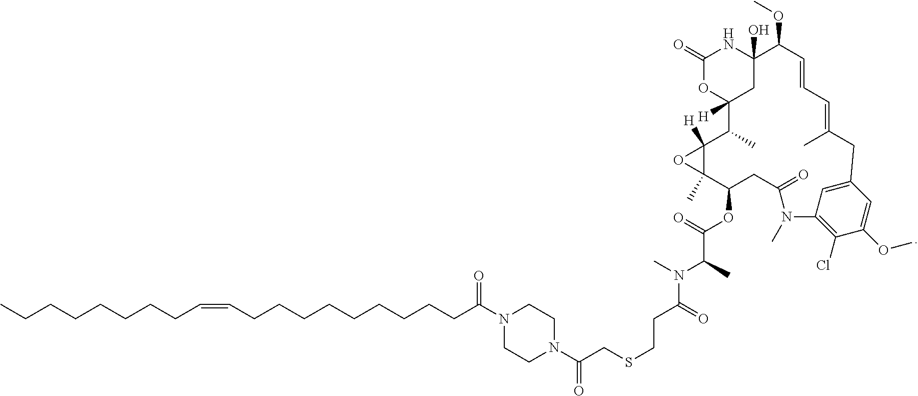 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

In another aspect, the subject has gastrointestinal cancer or precancer, or is suspected of developing gastrointestinal cancer or precancer.

Another embodiment described herein is a method for inhibiting the transition of a metaplastic cell to a dysplastic cell, the method comprising administering a therapeutically effective amount of 4-(2-chlorophenoxy)-N-(3-(methylcarbamoyl)phenyl)piperidine-1-carboxamide (A939572) to a cell or a subject in need thereof. In one aspect, the therapeutically effective amount of A939572 is from about 5 mg/kg to about 100 mg/kg. In another aspect, the therapeutically effective amount of A939572 is administered on a daily basis for about 1 day to about 6 months. In another aspect, the cell comprises a gastric epithelial cell. In another aspect, the subject has gastrointestinal cancer or precancer, or is suspected of developing gastrointestinal cancer or precancer.

Another embodiment described herein is a method for treating a metaplastic cell to reduce the incidence of carcinoma, the method comprising administering a therapeutically effective amount of 4-(2-chlorophenoxy)-N-(3-(methylcarbamoyl)phenyl)piperidine-1-carboxamide (A939572) to a cell or a subject in need thereof. In one aspect, the method inhibits the transition of the metaplastic cell to a dysplastic cell, thereby reducing the incidence of carcinoma in the cell or the subject in need thereof. In another aspect, the therapeutically effective amount of A939572 is from about 5 mg/kg to about 100 mg/kg. In another aspect, the therapeutically effective amount of A939572 is administered on a daily basis for about 1 day to about 6 months. In another aspect, the cell comprises a gastric epithelial cell. In another aspect, the subject has gastrointestinal cancer or precancer, or is suspected of developing gastrointestinal cancer or precancer.

Another embodiment described herein is a method of making a compound comprising cis-11-eicosenoic acid conjugated to 7-nitrobenzo[c][1,2,5]oxadiazol-4-amine (nitrobenzofurazan) or nitrobenzoselenadiazole:

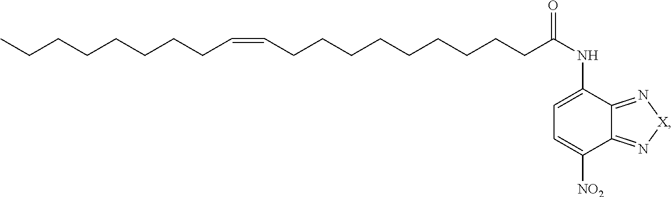

where X is O or Se; the method comprising: (a) combining cis-11-eicosenoic acid with dimethylformamide (DMF) solvent to create a solution; (b) adding N,N-diisopropylethylamine (DIPEA) to the solution; (c) adding 2-(1H-benzotriazole-1-yl)-1,1,3,3-tetramethyluronium hexafluorophosphate (HBTU) to the solution to create a reaction mixture; (d) mixing the reaction mixture; (e) adding nitrobenzofurazan or nitrobenzoselenadiazole to the reaction mixture to create a conjugate mixture; (f) mixing the conjugate mixture; and (g) one or more of diluting, washing, drying, or filtering the conjugate mixture to generate the compound. In one aspect, steps (a)-(e) are performed at about 0° C. and steps (f)-(g) are performed at room temperature. In another aspect, the conjugate mixture is diluted with ethyl acetate and washed with aqueous citric acid. In another aspect, the conjugate mixture is dried over MgSO₄. In another aspect, the method further comprises purifying the compound using column chromatography.

Another embodiment described herein is a method of making a compound comprising cis-11-eicosenoic acid conjugated to mertansine:

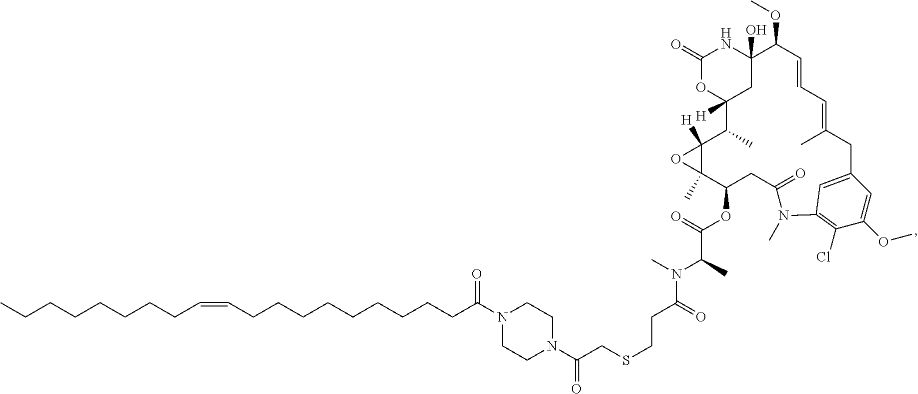 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

the method comprising: (a) combining mertansine carboxylic acid with dimethylformamide (DMF) solvent to create a solution; (b) adding hexafluorophosphate azabenzotriazole tetramethyl uranium (HATU) and N-methylmorpholine (NMM) to the solution to create a reaction mixture; (c) mixing the reaction mixture; (d) adding piperazine-conjugated cis-11-eicosenoic acid to the reaction mixture to create a conjugate mixture; (e) mixing the conjugate mixture; and (f) one or more of diluting, washing, drying, or filtering the conjugate mixture to generate the compound.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a graphic illustrating that the Gif-rtTA mice were crossed with the TetO-Cre and LSL-Kras^{G12D} mouse alleles to generate the Gif-rtTA; TetO-Cre; LSL-Kras^{G12D} (GCK) mouse model. GCK mice were administered doxycycline (DOX) in drinking water for 2 weeks to express the Gif-driven Cre enzyme and consequently induce active Kras expression.

FIG. 2A-D show the key stages of gastric carcinogenesis induced by Kras activation in gastric chief cells. FIG. 2A shows H&E-stained sections from the Gif-rtTA;TetO-Cre;Kras^{G12D} (GCK) mouse stomachs across multiple time points after doxycycline treatment: pyloric metaplasia (PM, 2-3 weeks), incomplete intestinal metaplasia (In-IM, 5-6 weeks), low-grade dysplasia/high-grade dysplasia (LGD/HGD, 8-10 weeks). FIG. 2B shows cytological features of dysplasia, observed in HGD, as indicated by yellow arrowheads. FIG. 2C shows the proportion of gland types in the GCK stomachs at multiple time points. 100 glands in the proximal region of the corpus were examined: 2-3 weeks (n=5), 5-6 weeks (n=8), 10-14 weeks (n=10). FIG. 2D shows measurement of gland width in GCK stomachs. A total of 100 glands were examined in each group, 3 mice per group. All IF data are representative of n=3 mice for each group and a total of 100 glands were examined in each group for quantitation. Hoechst was used for nuclear staining. White dotted boxes denote enlarged regions. Scale bars: 100 μm (FIG. 2A) and 25 μm (FIG. 2B). FIG. 2C shows mean±SEM. FIG. 2D shows mean±SD. One-way ANOVA with Tukey's multiple comparisons. ***p<0.001, ****p<0.0001.

FIG. 3A-E show that spatial and quantitative metabolic profiling reveals distinct metabolic patterns during carcinogenesis. FIG. 3A shows a schematic outline of the MALDI-IMS workflow to visualize the spatial distribution of metabolites in GCK stomachs. The mass spectrum (MS) images were obtained for each ion by plotting m/z intensity collected from each spot. FIG. 3B shows heat map displays of the relative values of average peak intensity of metabolites. FIG. 3C shows an overlay of MS images of monounsaturated fatty acid (MUFA; FFA 20:1). FIG. 3D shows a schematic overview of fatty acid (FA) desaturation and elongation. Palmitate obtained from de novo FA synthesis or diet. FAs can be converted intracellularly into other FA species by desaturation and elongation. FIG. 3E shows MS images of long-chain fatty acids in the GCK stomachs. The abundance of given metabolites in the corresponding MS image is normalized to 100%. Scale bar=500 μm.

FIG. 4A-F show specific expression of SCD1 in the GCK stomachs. FIG. 4A shows a schematic overview of key steps in central metabolic pathways with associated enzymes. FIG. 4B shows heat map displays of the relative mRNA levels of genes encoding metabolic enzymes or transporters in gastric organoid lines derived from GCK stomachs. n=3 biological replicates. FIG. 4C shows IF staining for SCD1 (green) and Ki-67 (red) in GCK stomachs. White dotted boxes indicate enlarged regions. White dotted lines identify the gland shape. FIG. 4D shows a schematic diagram of the SCD1 expression pattern in GCK stomachs. SCD1⁺ cells (green; cytoplasm), Ki-67⁺ cells (red; nucleus), and CD44v9⁺ cells (red; membrane). FIG. 4E shows IF staining for SCD1 (green) in normal or dysplastic organoids, derived from normal or GCK stomachs with high-grade dysplasia. All IF data are representative of n=3 mice for each group and Hoechst was used for nuclear staining. FIG. 4F shows phase-contrast images of dysplastic organoids treated with DMSO (vehicle), BMS-303141 (ACLY inhibitor; 1 μM), C75 (FASN inhibitor; 1 μM), A939572 (SCD inhibitor; 100 nM), or SC-26196 (FADS2 inhibitor; 1 μM) for 3 days. n≥3 biological replicates for each drug treatment. Scale bars: 100 μm (FIGS. 4C and 4E) and 1000 μm (FIG. 4F).

FIG. 5A-J show that SCD1 regulates dysplastic cell survival both in vivo and in vitro. FIGS. 5A and 5D show phase-contrast images of dysplastic or normal organoids treated with DMSO (vehicle) or 100 nM of A939572 (SCD inhibitor) at 0, 1, or 3 days after drug treatment. FIG. 5B shows quantitation of diameters of dysplastic or normal organoids before and after drug treatment. FIG. 5C shows H&E or IF staining for cleaved caspase-3 (CC-3, green) or cell proliferation (Ki-67, red) after drug treatment in dysplastic or normal organoids. n≥3 biological replicates for A939572 treatment. FIG. 5E shows H&E-stained corpus stomachs from GCK mice 2 weeks after vehicle (n=3) or A939572 (n=4) treatment. Yellow arrowheads indicate the dead cells. FIG. 5F shows the percentage of F4/80 positive area within the stromal areas in different regions of glands in the vehicle or A939572-treated GCK stomachs. FIG. 5G shows IF staining for CC-3 (green) in the vehicle or A939572-treated GCK stomachs. FIG. 5H shows quantitation of CC-3⁺ cells per 20× field of images. FIG. 5I shows IF staining for Terminal deoxynucleotidyl Transferase (TdT) dUTP Nick-End Labeling (TUNEL, green) in the vehicle or A939572-treated GCK stomachs. FIG. 5J shows quantitation of TUNEL⁺ cells per 20× field of images. Scale bars: 50 μm (FIG. 5E, right), 100 μm (FIGS. 5C, 5G, and 5I), 200 μm (FIG. 5E, left), and 1000 μm (FIGS. 5A and 5D). Hoechst was used for nuclear staining. Dotted boxes denote enlarged regions. All panels show mean±SD. Unpaired two-tailed Student's t test (I) or One-way ANOVA with Tukey's multiple comparisons (FIGS. 5B, 5H, and 5J). **p<0.01, ***p<0.001, ****p<0.0001.

FIG. 6A-G show the common feature of SCD expression in dysplasia lesions across the GI tract carcinogenesis. FIG. 6A shows immunohistochemistry (IHC) staining for SCD in adjacent normal (n=4), IM (n=11), LGD (n=7), HGD (n=5), and intestinal- or diffuse-type gastric cancer (GC) tissues (n=89). FIG. 6B shows IHC staining for SCD in adjacent normal (n=9), Barrett's esophagus (BE, n=13), dysplasia (n=6), and esophageal adenocarcinoma (ADC, n=11). FIG. 6C-D show the IHC H-score of SCD in human stomach tissues (FIG. 6C), and esophageal tissues (FIG. 6D). FIG. 6E shows IHC staining for SCD in adjacent normal (n=16), acinar-to-ductal metaplasia (ADM, n=14), mucinous cystic neoplasia (MCN, n=5), intraductal papillary mucinous neoplasia (IPMN, n=14), pancreatic intraepithelial neoplasia (PanIN, n=18), and pancreatic adenocarcinoma (PDAC, n=10). FIG. 6F shows the IHC H-score of SCD in human pancreatic tissues. FIG. 6G shows a schematic illustration of the proposed model. Scale bars: 50 μm (FIGS. 6A and 6B) and 100 μm (FIG. 6C). All panels show mean±SD. Unpaired two-tailed Student's t test (FIG. 6G) or One-way ANOVA with Tukey's multiple comparisons. **p<0.01, ***p<0.001, ****p<0.0001.

FIG. 7A-J show that dysplastic cells actively use the MUFA synthesized from SCD-dependent FA desaturation as a key energy source. FIG. 7A shows a schematic overview of FA elongation and desaturation for eicosenoic acid (EA; 20:1n9) or docosatetraenoic acid (DA; 22:4n6). FIG. 7B shows the chemical structure of synthesized NBD (nitrobenzoxadiazole; nitrobenzofurazan)-conjugated EA (20:1n9). FIG. 7C-E show phase-contrast, H&E images (FIG. 7C), quantitation of organoid diameters (FIG. 7D), and live/dead (Calcein AM/Ethidium homodimer-1 [EthD-1]) cell staining (FIG. 7E) of dysplastic organoids treated with DMSO or A939572 in medium with supplementation of EA or DA for 3 days. FIG. 7F shows confocal images of dysplastic cell monolayers incubated with NBD (green) fluorescence conjugated EA (20:1n9) at 6 h, 24 h, and 48 h incubation and then stained with 100 nM of MitoTracker™ Red CMXRos (Mito). Hoechst was used for nuclear staining (Nuc). White dotted boxes indicate enlarged regions. FIG. 7G shows relative mRNA expression levels of fatty acid oxidation (FAO) genes in gastric organoid lines from GCK stomachs. FIG. 7H shows a schematic for co-treatment of FAO inhibitor (Perhexiline 10 μM) treated with DMSO or A939572 in dysplastic organoids with supplementation of EA for 3 days. FIG. 7I-J show phase-contrast images (FIG. 7I) and quantitation of diameters (FIG. 7J) of dysplastic organoids after the co-treatment. n≥3 biological replicates. Scale bars: 20 μm (FIG. 7F), 100 μm (FIGS. 7C, bottom; and 7E), and 1000 μm (FIGS. 7C, top; and 7I). All panels show mean±SD. One-way ANOVA with Tukey's multiple comparisons. **p<0.01, ***p<0.001, ****p<0.0001.

FIG. 8A-B show the synthesis of NBD (FIG. 8A) or NBD fluorescence-conjugated metabolite (FIG. 8B). Chemical structure and NMR profile of the synthesized NBD (FIG. 8A) or NBD fluorescence-conjugated eicosenoic acid (20:1n9) (FIG. 8B).

FIG. 9A-B show cells treated with EA-DM1 (cis-11-eicosenoic acid conjugated to mertansine (DM1)). FIG. 9A shows AGS and NCI-N87 cell lines treated with either DMSO or EA-DM1 (100 nM) for 2 days prior to being imaged. DMSO was used as a vehicle control. FIG. 9B shows NIH-3T3 and AGS cell lines were treated with either DMSO or EA-DM1 (5 nM) for 2 days prior to being imaged. The NIH-3T3 cell line was used as a negative control, and mouse normal cells and DMSO were used as a vehicle control.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 20-30° C., including all integers and endpoints within the specified range. In another aspect, “room temperature” refers a temperature of about 20-30° C.; about 22-30° C.; about 25-30° C.; about 27-30° C.; about 20-22° C.; about 20-25° C.; about 20-27° C.; about 22-25° C.; about 22-27° C.; about 25-27° C.; about 20° C.±10%; about 22° C.±10%; about 25° C.±10%; about 27° C.±10%; −20° C., −22° C., −25° C., or −27° C., at standard atmospheric pressure.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “metaplasia” or “metaplastic” refers to the transformation of one differentiated cell type to another differentiated cell type that does not normally occur in the tissue in which it is found. The change from one type of cell to another may be part of a normal maturation process or may be caused by some type of abnormal stimulus.

As used herein, “dysplasia” or “dysplastic” refers to any of various types of abnormal growth or development of a cell type, tissue, or organ, and the abnormal histology or anatomical structure(s) resulting from such abnormal growth or development. As disclosed herein, dysplasia may be a condition in which cells have abnormal cellular and architectural changes and is the focal neoplastic lesion, which has the highest risk of cancer development.

As used herein, “cancer” or “cancerous” refers to malignant and invasive epithelial adenocarcinoma characterized by rapid and uncontrolled cell growth and proliferation. Cancerous adenocarcinoma cells may develop from the malignant transformation of metaplastic and dysplastic epithelial cells and can spread locally or metastasize through the bloodstream and lymphatic system to other parts of the body. In some embodiments described herein, a subject may be treated for cancer by administering to the subject one or more compounds as described herein. In one aspect, the subject may have gastrointestinal cancer, or may be suspected of developing gastrointestinal cancer.

As used herein, “precancer” or “precancerous” refers to non-malignant and non-invasive metaplastic epithelial cells that have yet to undergo transformation into dysplastic or adenocarcinoma cells. Precancerous metaplasia may occur when the mucosa of the stomach (i.e., gastric mucosa) is replaced with intestinal epithelium. Multiple types of precancerous lesions exist in human epithelial cell carcinogenesis and the terms, order, and mutational signatures are different between organs. In pancreatic cancer, for example, three predominant types including mucinous cystic neoplasia, intraductal papillary mucinous neoplasia, and pancreatic intraepithelial neoplasia are defined as precancerous lesions. In some embodiments described herein, a subject may be treated for precancer by administering to the subject one or more compounds as described herein. In one aspect, the subject may have gastrointestinal precancer, or may be suspected of developing gastrointestinal precancer, including precancerous lesions.

Epithelial carcinogenesis develops within a sequential carcinogenic cascade from precancerous metaplasia to dysplasia and adenocarcinoma, and oncogenic gene activation can drive the process. Metabolic reprogramming is considered a key mechanism that regulates cancer cell growth and proliferation. To define metabolic dynamics during gastric carcinogenesis, imaging mass spectrometry was performed using a mouse model of metaplasia and dysplasia following Kras activation in zymogen-secreting chief cells. It was determined that metabolic reprogramming from glycolysis to fatty acid metabolism occurs during precancerous cell lineage conversion into dysplastic cells. Altered fatty acid desaturation through stearoyl-CoA desaturase (SCD) produces a novel eicosenoic acid, which fuels dysplastic cell hyperproliferation and survival. These results indicate that oncogenic metabolic rewiring serves as a driver of malignant transformation of precancerous cells in carcinogenesis.

As disclosed herein, a study was focused on gastric carcinogenesis and metabolism which might be involved in the key transition between metaplasia and dysplasia during the gastric carcinogenesis using a novel mouse model that induces Kras activation in zymogen-granule secreting chief cells. Imaging mass spectrometry was used to investigate dynamic changes of metabolite accumulation in the carcinogenic process and identified a novel fatty acid metabolic pathway which utilizes an eicosenoic acid as a major source of energy during precancerous metaplasia progression to dysplasia.

Suitable small-sized multi-colored fluorophores for use in the disclosed invention may include those described in Benson et al., SCOTfluors: small, conjugatable, orthogonal, and tunable fluorophores for in vivo imaging of cell metabolism, Angew Chem Int Ed Engl., 131(21): 6985-6989 (2019), the entire contents of which are incorporated by reference into the specification.

One embodiment described herein is a compound of formula (I):

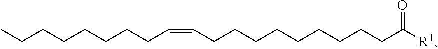

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine. In one aspect, the compound is:

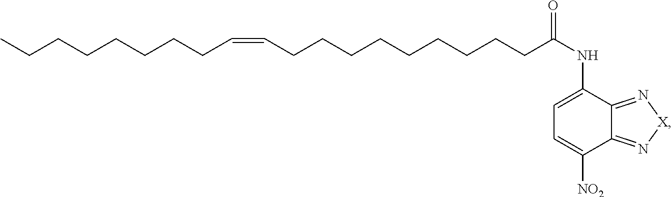

where X is O or Se.

Another embodiment described herein is a compound of formula (I):

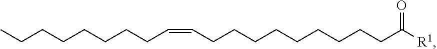

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

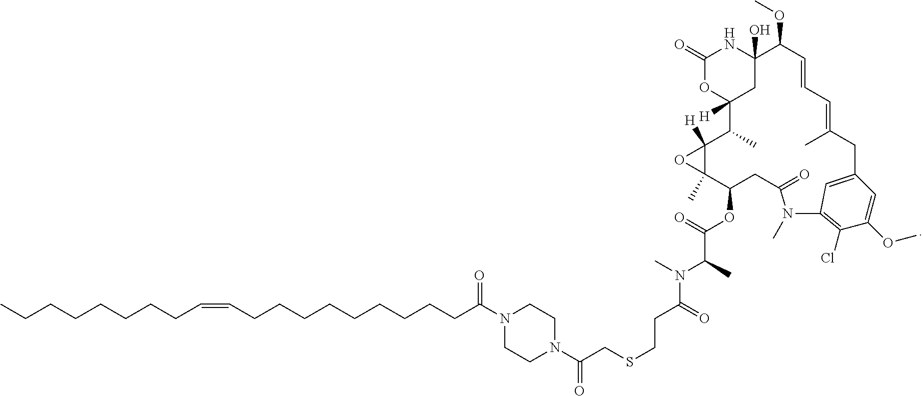 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method for detecting epithelial cell dysplasia, the method comprising: contacting an epithelial cell with a compound as described herein; incubating for a period of time; irradiating the epithelial cell with ultraviolet light, whereby a dysplastic epithelial cell will fluoresce; and imaging the fluorescent dysplastic epithelial cell. In one aspect, imaging comprises positron emission tomography (PET) imaging, mass spectrometry imaging, immunofluorescence imaging, fluorescence molecular endoscopy, or fluorescence-guided intraluminal endoscopy. In another aspect, the epithelial cell comprises a gastric epithelial cell.

Another embodiment described herein is a research tool for detecting epithelial dysplasia, the research tool comprising a compound of formula (I):

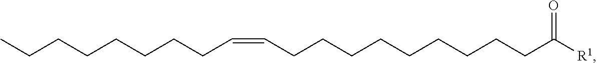

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine. In one aspect, the compound is:

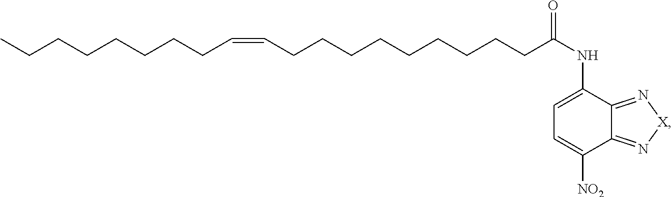

where X is O or Se.

Another embodiment described herein is a research tool comprising a compound of formula (I):

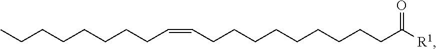

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

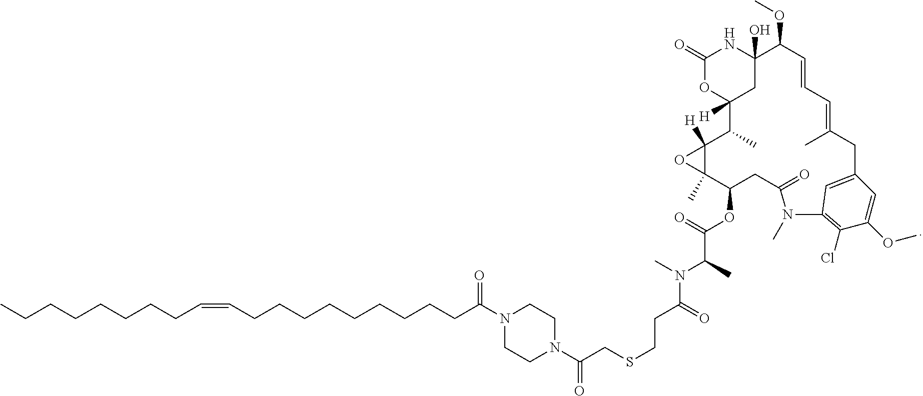 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method of inhibiting cancer or precancer in a cell or a subject in need thereof, the method comprising administering to the cell or the subject a therapeutically effective amount of a compound of formula (I):

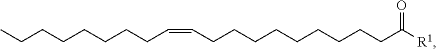

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

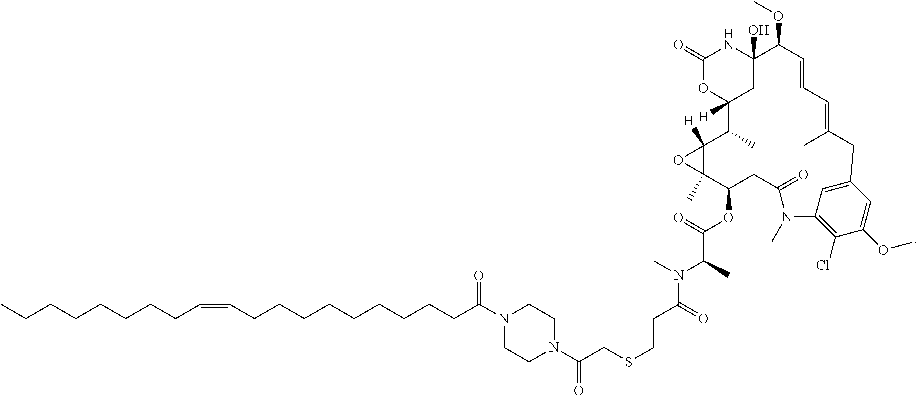 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Another embodiment described herein is a method of treating a subject having cancer or precancer, the method comprising administering to the subject a therapeutically effective amount of a compound of formula (I):

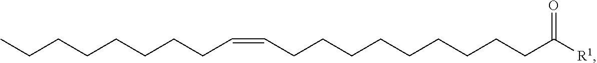

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90. In one aspect, the compound is:

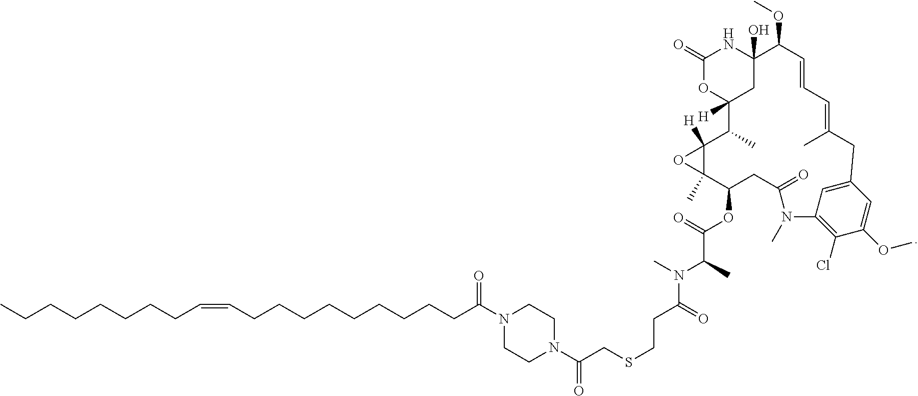 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

In another aspect, the subject has gastrointestinal cancer or precancer, or is suspected of developing gastrointestinal cancer or precancer.

Another embodiment described herein is a method for inhibiting the transition of a metaplastic cell to a dysplastic cell, the method comprising administering a therapeutically effective amount of 4-(2-chlorophenoxy)-N-(3-(methylcarbamoyl)phenyl)piperidine-1-carboxamide (A939572) to a cell or a subject in need thereof. In one aspect, the therapeutically effective amount of A939572 is from about 5 mg/kg to about 100 mg/kg. In another aspect, the therapeutically effective amount of A939572 is administered on a daily basis for about 1 day to about 6 months. In another aspect, the cell comprises a gastric epithelial cell. In another aspect, the subject has gastrointestinal cancer or precancer, or is suspected of developing gastrointestinal cancer or precancer.

Another embodiment described herein is a method for treating a metaplastic cell to reduce the incidence of carcinoma, the method comprising administering a therapeutically effective amount of 4-(2-chlorophenoxy)-N-(3-(methylcarbamoyl)phenyl)piperidine-1-carboxamide (A939572) to a cell or a subject in need thereof. In one aspect, the method inhibits the transition of the metaplastic cell to a dysplastic cell, thereby reducing the incidence of carcinoma in the cell or the subject in need thereof. In another aspect, the therapeutically effective amount of A939572 is from about 5 mg/kg to about 100 mg/kg. In another aspect, the therapeutically effective amount of A939572 is administered on a daily basis for about 1 day to about 6 months. In another aspect, the cell comprises a gastric epithelial cell. In another aspect, the subject has gastrointestinal cancer or precancer, or is suspected of developing gastrointestinal cancer or precancer.

Another embodiment described herein is a method of making a compound comprising cis-11-eicosenoic acid conjugated to 7-nitrobenzo[c][1,2,5]oxadiazol-4-amine (nitrobenzofurazan) or nitrobenzoselenadiazole:

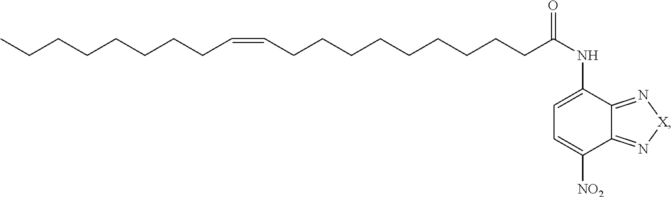

where X is O or Se; the method comprising: (a) combining cis-11-eicosenoic acid with dimethylformamide (DMF) solvent to create a solution; (b) adding N,N-diisopropylethylamine (DIPEA) to the solution; (c) adding 2-(1H-benzotriazole-1-yl)-1,1,3,3-tetramethyluronium hexafluorophosphate (HBTU) to the solution to create a reaction mixture; (d) mixing the reaction mixture; (e) adding nitrobenzofurazan or nitrobenzoselenadiazole to the reaction mixture to create a conjugate mixture; (f) mixing the conjugate mixture; and (g) one or more of diluting, washing, drying, or filtering the conjugate mixture to generate the compound. In one aspect, steps (a)-(e) are performed at about 0° C. and steps (f)-(g) are performed at room temperature. In another aspect, the conjugate mixture is diluted with ethyl acetate and washed with aqueous citric acid. In another aspect, the conjugate mixture is dried over MgSO₄. In another aspect, the method further comprises purifying the compound using column chromatography.

Another embodiment described herein is a method of making a compound comprising cis-11-eicosenoic acid conjugated to mertansine:

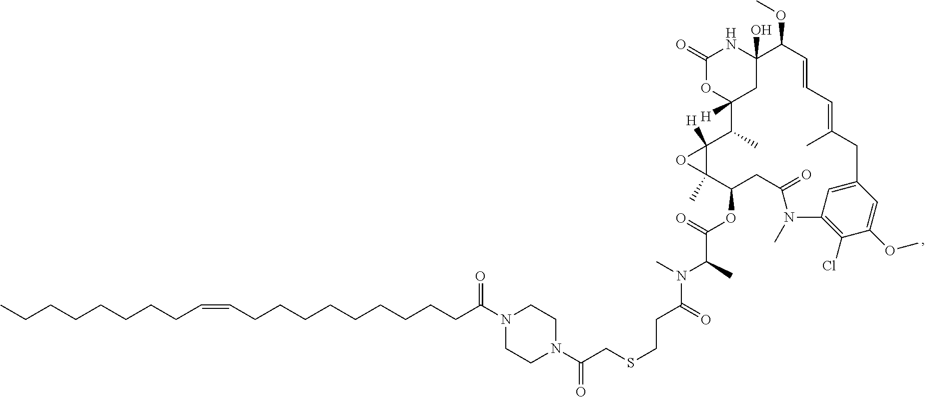 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

the method comprising: (a) combining mertansine carboxylic acid with dimethylformamide (DMF) solvent to create a solution; (b) adding hexafluorophosphate azabenzotriazole tetramethyl uranium (HATU) and N-methylmorpholine (NMM) to the solution to create a reaction mixture; (c) mixing the reaction mixture; (d) adding piperazine-conjugated cis-11-eicosenoic acid to the reaction mixture to create a conjugate mixture; (e) mixing the conjugate mixture; and (f) one or more of diluting, washing, drying, or filtering the conjugate mixture to generate the compound.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A compound of formula (I):

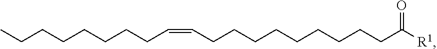

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine.
Clause 2. The compound of clause 1, wherein the compound is:

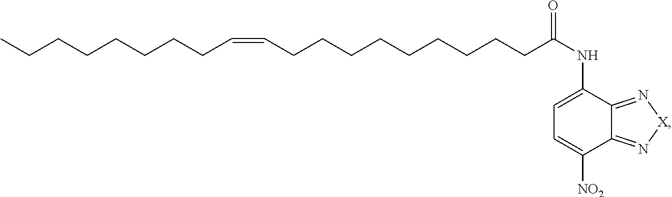

where X is O or Se.
Clause 3. A compound of formula (I):

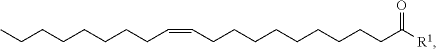

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.
Clause 4. The compound of clause 3, wherein the compound is:

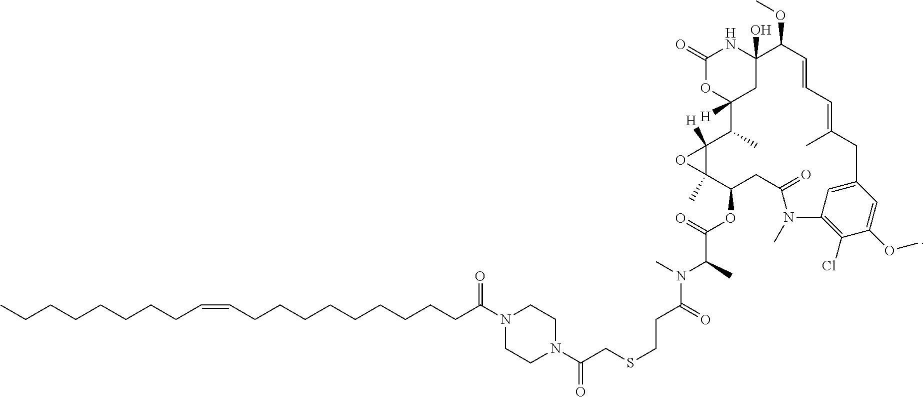 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Clause 5. A method or detecting epithelial cell dysplasia, the method comprising:
   contacting an epithelial cell with the compound of clause 1;
   incubating for a period of time;
   irradiating the epithelial cell with ultraviolet light, whereby a dysplastic epithelial cell will fluoresce; and
   imaging the fluorescent dysplastic epithelial cell.
Clause 6. The method of clause 5, wherein imaging comprises positron emission tomography (PET) imaging, mass spectrometry imaging, immunofluorescence imaging, fluorescence molecular endoscopy, or fluorescence-guided intraluminal endoscopy,
Clause 7. The method of clause 5 or 6, wherein the epithelial cell comprises a gastric epithelial cell.
Clause 8. A research tool for detecting epithelial dysplasia, the research tool comprising a compound of formula (I):

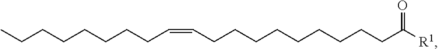

wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine.
Clause 9. The research tool of clause 8, wherein the compound is:

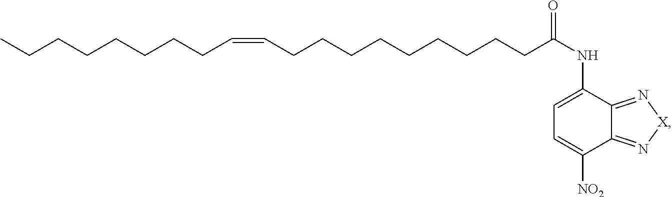

where X is O or Se.
Clause 10. A research tool comprising a compound of formula (I):

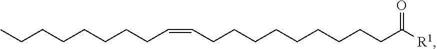

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.
Clause 11. The research tool of clause 10, wherein the compound is:

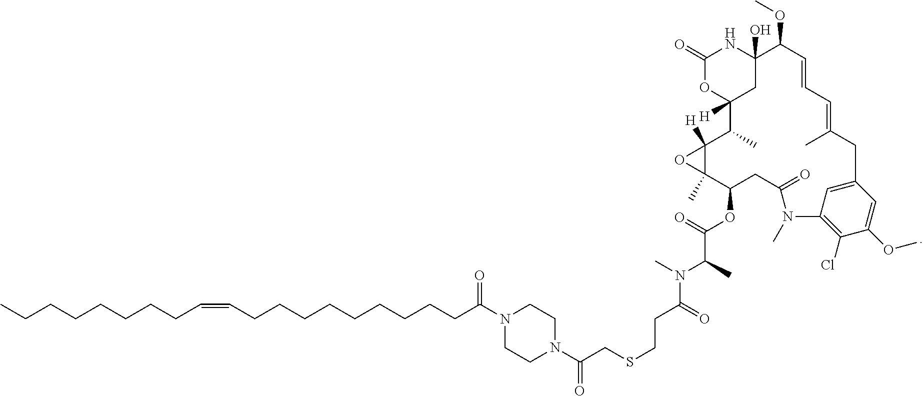 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Clause 12. A method of inhibiting cancer or precancer in a cell or a subject in need thereof, the method comprising administering to the cell or the subject a therapeutically effective amount of a compound of formula (I):

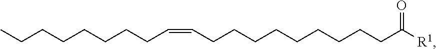

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.
Clause 13. The method of clause 12, wherein the compound is:

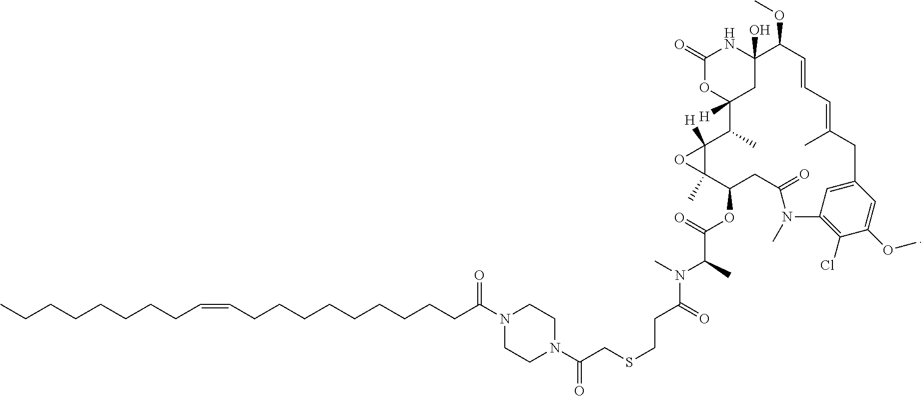 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Clause 14. A method of treating a subject having cancer or precancer, the method comprising administering to the subject a therapeutically effective amount of a compound of formula (I):

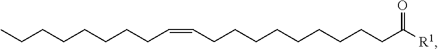

wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.
Clause 15. The method of clause 14, wherein the compound is:

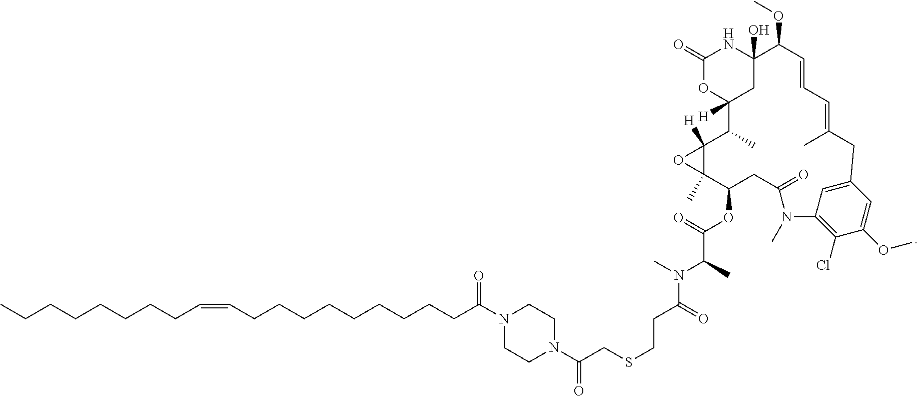 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

Clause 16. The method of clause 14 or 15, wherein the subject has gastrointestinal cancer or precancer, or is suspected of developing gastrointestinal cancer or precancer.
Clause 17. A method for inhibiting the transition of a metaplastic cell to a dysplastic cell, the method comprising administering a therapeutically effective amount of 4-(2-chlorophenoxy)-N-(3-(methylcarbamoyl)phenyl)piperidine-1-carboxamide (A939572) to a cell or a subject in need thereof.
Clause 18. The method of clause 17, wherein the therapeutically effective amount of A939572 is from about 5 mg/kg to about 100 mg/kg.
Clause 19. The method of clause 17 or 18, wherein the therapeutically effective amount of A939572 is administered on a daily basis for about 1 day to about 6 months.
Clause 20. The method of any one of clauses 17-19, wherein the cell comprises a gastric epithelial cell.
Clause 21. The method of any one of clauses 17-20, wherein the subject has gastrointestinal cancer or precancer, or is suspected of developing gastrointestinal cancer or precancer.
Clause 22. A method for treating a metaplastic cell to reduce the incidence of carcinoma, the method comprising administering a therapeutically effective amount of 4-(2-chlorophenoxy)-N-(3-(methylcarbamoyl)phenyl)piperidine-1-carboxamide (A939572) to a cell or a subject in need thereof.
Clause 23. The method of clause 22, wherein the method inhibits the transition of the metaplastic cell to a dysplastic cell, thereby reducing the incidence of carcinoma in the cell or the subject in need thereof.
Clause 24. The method of clause 22 or 23, wherein the therapeutically effective amount of A939572 is from about 5 mg/kg to about 100 mg/kg.
Clause 25. The method of any one of clauses 22-24, wherein the therapeutically effective amount of A939572 is administered on a daily basis for about 1 day to about 6 months.
Clause 26. The method of any one of clauses 22-25, wherein the cell comprises a gastric epithelial cell.
Clause 27. The method of any one of clauses 22-26, wherein the subject has gastrointestinal cancer or precancer, or is suspected of developing gastrointestinal cancer or precancer.
Clause 28. A method of making a compound comprising cis-11-eicosenoic acid conjugated to 7-nitrobenzo[c][1,2,5]oxadiazol-4-amine (nitrobenzofurazan) or nitrobenzoselenadiazole:

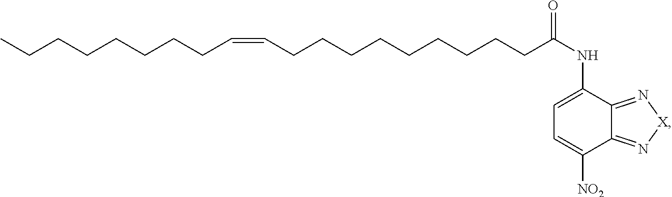

where X is O or Se;
   the method comprising:
   (a) combining cis-11-eicosenoic acid with dimethylformamide (DMF) solvent to create a solution;
   (b) adding N,N-diisopropylethylamine (DIPEA) to the solution;
   (c) adding 2-(1H-benzotriazole-1-yl)-1,1,3,3-tetramethyluronium hexafluorophosphate (HBTU) to the solution to create a reaction mixture;
   (d) mixing the reaction mixture;
   (e) adding nitrobenzofurazan or nitrobenzoselenadiazole to the reaction mixture to create a conjugate mixture;
   (f) mixing the conjugate mixture; and
   (g) one or more of diluting, washing, drying, or filtering the conjugate mixture to generate the compound.
Clause 29. The method of clause 28, wherein steps (a)-(e) are performed at about 0° C. and steps (f)-(g) are performed at room temperature.
Clause 30. The method of clause 28 or 29, wherein the conjugate mixture is diluted with ethyl acetate and washed with aqueous citric acid.
Clause 31. The method of any one of clauses 28-30, wherein the conjugate mixture is dried over MgSO₄.
Clause 32. The method of any one of clauses 28-31, further comprising purifying the compound using column chromatography.
Clause 33. A method of making a compound comprising cis-11-eicosenoic acid conjugated to mertansine:

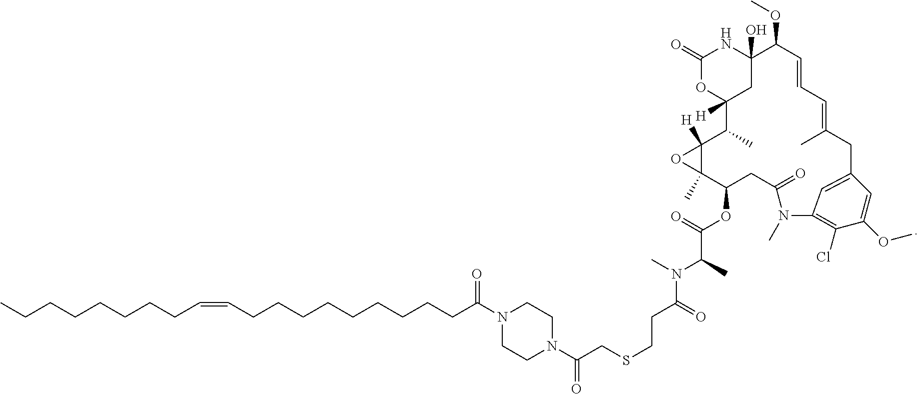 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

the method comprising:
(a) combining mertansine carboxylic acid with dimethylformamide (DMF) solvent to create a solution;
(b) adding hexafluorophosphate azabenzotriazole tetramethyl uranium (HATU) and N-methylmorpholine (NMM) to the solution to create a reaction mixture;
(c) mixing the reaction mixture;
(d) adding piperazine-conjugated cis-11-eicosenoic acid to the reaction mixture to create a conjugate mixture;
(e) mixing the conjugate mixture; and
(f) one or more of diluting, washing, drying, or filtering the conjugate mixture to generate the compound.

### EXAMPLES

### Example 1

### Materials and Methods

### Mice

All experiments involving mice used in this study followed protocols approved by the Institutional Animal Care and Use Committees of Vanderbilt University Medical Center. Littermates or age-matched mice were randomly allocated to experimental or control groups. Animal weights were recorded at initiation of experiment and at the time of euthanasia. To generate the Gif-rtTA; TetO-Cre; Kras^{G12D} (GCK) mice, the Gif-rtTA mice were crossed with TetO-Cre mice and Lox-Stop-Lox (LSL)-Kras^{G12D} mice (no. 006224 and 008179, Jackson Laboratories). For the induction of Cre-mediated recombination, 6-week-old mice were administered with doxycycline water at a concentration of 1 mg/mL for 2 weeks. Mice were sacrificed at 2 to 14 weeks after doxycycline treatment for histological examination. For in vivo A939572 treatment, A939572 (HY-50709; MedChemExpress) was dissolved in dimethyl sulfoxide (DMSO) as a 100 mg/mL stock and stored at −80° C. in aliquots. The A939572 was administered daily for two weeks by intraperitoneal injection (20 mg/kg diluted in corn oil) to mice at 6 weeks after doxycycline treatment. Mice were sacrificed within a day after the last A939572 treatment.

### Human Tissue Acquisition and Sample Preparation

Details on the clinicopathological characteristics of human subject are provided in Table 1. In total, 6 human tissue array sets (5 gastric tissue arrays and 1 esophageal tissue array) were constructed with specimens from the patients who underwent curative endoscopic or surgical resection at Seoul National University Hospital or Jeju National University Hospital from 2010 to 2021 in South Korea. Briefly, representative regions were selected from H&E-stained slides through histologic examination. The tissue cores with 4 mm in diameter were obtained from individual paraffin blocks and arranged in a recipient paraffin block using a trephine apparatus (SuperBioChips Laboratories, Seoul, Korea). Tissue array construction was approved by the Institutional Review Board of Seoul National University Hospital and Jeju National University Hospital, respectively. Informed consent was waived by the Institutional Review Board due to the retrospective nature of the study. 6 esophageal dysplasia tissues were provided by the NCI Cooperative Human Tissue Network (CHTN) (221092). 30 pancreatic tissues obtained from 27 patients who underwent surgical resection at Vanderbilt University Medical Center.

TABLE 1
Human Sample Clinicopathological Characteristics
Esophageal tissue

| | | Normal | BE | Dysplasia | | ADC | |
|---|---|---|---|---|---|---|---|
| Sample No. | | 9 | 13 | 6 | | 11 | |
| Age, Mean ± SD | | — | — | 71 ± 8 | | 57 ± 13 | |
| Gender, | Male | — | — | 6 | (100) | 10 | (91) |
| n (%) | Female | — | — | 0 | (0) | 1 | (9) |

| Size (mm), | | — | — | — | 2.7 ± 1.5 | |
|---|---|---|---|---|---|---|
| Mean ± SD | | | | | | |
| Histology | WD | — | — | — | 4 | (36) |
| | MD | — | — | — | 5 | (46) |
| | PD | — | — | — | 1 | (9) |
| | Muc | — | — | — | 1 | (9) |
| TNM | I | — | — | — | 7 | (64) |
| stage | II | — | — | — | 1 | (9) |
| | III | — | — | — | 3 | (27) |
| | IV | — | — | — | 0 | (0) |

Gastric Tissue

| | | Normal | IM | LGD | | HGD | | EGC | | GC | |
|---|---|---|---|---|---|---|---|---|---|---|---|
| Sample No. | | 4 | 11 | 7 | | 5 | | 91 | | 89 | |
| Age, Mean ± SD | | — | — | 68 ± 9 | | 68 ± 12 | | 65 ± 11 | | 71 ± 13 | |
| Gender, n | Male | — | — | 5 | (71) | 5 | (100) | 62 | (68) | 53 | (60) |
| (%) | Female | — | — | 2 | (29) | 0 | (0) | 29 | (32) | 36 | (40) |

| | | | | | | | | | | |
|---|---|---|---|---|---|---|---|---|---|---|
| Location, n | Body | — | — | 0 | (0) | 1 | (20) | 44 | (48) | — |
| (%) | Antrum | — | — | 7 | (100) | 4 | (80) | 47 | (52) | — |

| Size (mm), | | — | — | 1.0 ± 0.4 | 1.6 ± 1.1 | 29 ± 20 | | 56 ± 32 | |
|---|---|---|---|---|---|---|---|---|---|
| Mean ± SD | | | | | | | | | |
| Histology | WD | — | — | — | — | 23 | (25) | 0 | (0) |
| | MD | — | — | — | — | 53 | (58) | 28 | (32) |
| | PD | — | — | — | — | 8 | (9) | 44 | (49) |
| | PCC | — | — | — | — | 7 | (8) | 17 | (19) |
| | (SRCC) | | | | | | | | |
| TNM | I | — | — | — | — | 91 | (100) | 33 | (37) |
| stage | II | — | — | — | — | | | 23 | (26) |
| | III | — | — | — | — | | | 33 | (37) |
| | IV | — | — | — | — | | | 0 | (0) |

Pancreatic tissue

| | | | | | | | | PDAC | | | |
|---|---|---|---|---|---|---|---|---|---|---|---|
| | | Normal | ADM | MCN | | IPMN | | PanIN-LG | | PanIN-HG | |
| Sample No. | | 16 | 14 | 5 | | 14 | | 5 | | 5 | |
| Age, Mean ± SD | | — | — | 57 ± 19 | | 72 ± 6 | | 60 ± 13 | | 65 ± 12 | |
| Gender, | Male | — | — | 4 | (80) | 7 | (50) | 2 | (40) | 0 | (0) |
| n (%) | Female | — | — | 1 | (20) | 7 | (50) | 3 | (60) | 5 | (100) |
| Location, | Head | — | — | 0 | (0) | 12 | (86) | 4 | (80) | 5 | (100) |
| n (%) | Body/Tail | — | — | 5 | (100) | 2 | (14) | 1 | (20) | 0 | (0) |

| Size of invasive | | — | — | — | 27 ± 26 | 38 ± 18 | | 20 ± 0 | |
|---|---|---|---|---|---|---|---|---|---|
| component (mm) | | | | | | | | | |
| Mean ± SD | | | | | | | | | |
| Size of tumor | | — | — | 49 ± 30 | 32 ± 17 | — | | — | |
| (mm) Mean ± SD | | | | | | | | | |
| Histology | WD | — | — | — | — | 0 | (0) | 0 | (0) |
| | MD | — | — | — | — | 3 | (60) | 1 | (20) |
| | PD | — | — | — | — | 2 | (40) | 4 | (80) |
| | Undiffer | — | — | — | — | 0 | (0) | 0 | (0) |
| TNM | I | — | — | — | — | 0 | (0) | 0 | (0) |
| stage | II | — | — | — | — | 5 | (100) | 5 | (100) |
| | III | — | — | — | — | 0 | (0) | 0 | (0) |
| | IV | — | — | — | — | 0 | (0) | 0 | (0) |

Abbreviations: BE, Barrett's esophagus; ADC, adenocarcinoma; SD, standard deviation; GEJ, gastroesophageal junction; WD, well differentiated; MD, moderately differentiated; PD, poorly differentiated; Muc, mucinous; TNM, Tumor, node and metastasis; IM, intestinal metaplasia; LGD, low-grade dysplasia; HGD, high-grade dysplasia; EGC, early gastric cancer; GC, gastric cancer; PCC, poorly cohesive carcinoma; SRCC, signet-ring cell carcinoma; ADM, acinar-to-ductal metaplasia; MCN, mucinous cystic neoplasm; IPMN, intraductal papillary mucinous neoplasm; PDAC, pancreatic ductal adenocarcinoma; PanIN, pancreatic intraepithelial neoplasia; Undiffer, Undifferentiated.

### Histology and Immunostaining

Mouse tissues were isolated and fixed overnight in 4% paraformaldehyde, embedded in paraffin. Paraffin-embedded sections were sectioned at a thickness of five-micrometer and stained with hematoxylin and eosin (H&E). For immunostaining, unstained paraffin tissue sections were deparaffinized in HistoClear (HS-200, National Diagnostics) and rehydrated through a serial dilution of ethanol. Antigen retrieval was performed in target retrieval solution (S1699 or S2367, Agilent) using a pressure cooker for 15 minutes. Sections were rinsed in dH2O and incubated with Serum-free Protein Block Solution (X0909, Agilent) for 1.5 hours at room temperature (RT). Sections were additionally incubated with Mouse on Mouse Blocking Reagent (MKB-2213-1, Vector Laboratories) for 30 min at RT to block nonspecific staining with primary antibodies generated in mice on mouse tissues. Primary antibodies were diluted in Antibody Diluent (S3022, Agilent) and applied to sections overnight at 4° C. (Table 2) in humidified chamber.

TABLE 2
Primary Antibodies for Immunostaining

| Antibody | Species | Vender, Catalog# | Dilution |
|---|---|---|---|
| AQP5 | Rabbit | Sigma-Aldrich, HPA065008 | 1:500 |
| CD133 (Prominin-1) | Rat | Invitrogen, 14-1331-82 | 1:100 |
| CD68 | Mouse | Santacruz, sc-20060 | 1:500 |
| CD44v9 | Rat | Cosmo Bio, LKG-M002 | 1:15000 |
| CEACAM5 for mouse | Mouse | ABclonal, A18131 | 1:1000 |
| CLDN3 | Rabbit | ABclonal, A2946 | 1:1000 |
| Cleaved Caspase-3 (Asp175) | Rabbit | Cell signaling, 9661 | 1:500 |
| F4/80 | Rat | Invitrogen, MF48000 | 1:500 |
| Ki-67 for mouse | Rat | BioLegend, 652402 | 1:500 |
| Ki-67 for human | Rat | LSBio, LS-C338537 | 1:500 |
| Ki-67 for mouse/human | Rabbit | Abcam, ab16667 | 1:500 |
| Phospho-Histone H3 (Ser10) | Mouse | Invitrogen, MA5-15220 | 1:500 |
| SCD1 for mouse | Rabbit | Cell signaling, 2794 | 1:200 |
| SCD for human | Mouse | Abcam, ab19862 | 1:5000 |
| TFF3 | Rabbit | Gift from Daniel K. Podolsky | 1:1000 |
| TROP2 for mouse | Goat | R&D Systems, AF1122 | 1:500 |
| TROP2 for human | Goat | R&D Systems, AF650 | 1:500 |

For immunofluorescence, paraffin sections were washed with 1×PBS three times and incubated with Alexa-conjugated secondary antibodies (Life Technologies, 1:500) for 1 hour at RT. Sections were washed with 1×PBS three times and incubated with Hoechst 33342 (62249, Thermo Fisher Scientific, 1:5000) for 5 min at RT. The sections were then washed with 1×PBS three times and mounted with Prolong gold anti-fade reagent (P36934, Thermo Fisher Scientific). Images were captured on a Zeiss Axio Imager M2 microscope with Axiovision digital imaging system at 20× magnification. For immunohistochemistry, paraffin sections were washed with 1×PBS three times and incubated with HRP-conjugated secondary antibodies (MP-7402, Vector Laboratories) for 15 min at RT. Sections were washed with 1×PBS three times and incubated with Envision+ Detection System Peroxidase/DAB substrate (Dako). Sections were then washed with dH₂O and counterstained Mayer's Hematoxylin (MHS32, Sigma-Aldrich). The sections were dehydrated and mounted with aqueous mounting medium. The sections were scanned on an SCN400 Slide Scanner (Leica Biosystems) at 20× magnification. TUNEL staining was performed using the Click-iT Plus TUNEL assay kit, according to the manufacturer's protocol (C10618, Life Technologies).

### Histopathologic Analysis and Quantitation

H&E-stained sections were scanned in SCN400 Slide Scanner and analyzed by Aperio ImageScope software (Leica Biosystems). Gland types were classified using the same diagnostic criteria used for human tissue sections. Briefly, the gland representing foveolar hyperplasia without goblet cells and mucous metaplasia at the base was diagnosed as a pyloric metaplastic gland, while intestinal metaplasia was determined by presence of basophilic mucin droplet-containing cells in the foveolar region. Low-grade dysplasia was characterized by morphological characteristics including crowed tubular or glandular structure composed of pseudostratified columnar cells with hyperchromatic nuclei (Ref). Compared with others, high-grade dysplastic glands showed structural complexity and were lined by the cells with severe cellular and nuclear atypia and loss of polarity. Mitotic figures in the entire gland were more frequently observed in high-grade dysplastic glands. Gland width was measured on both ends of the transitioning area per gland using the Aperio ImageScope software. A total of 100 glands from 3 mice at each stage were measured in each tissue section. At least 5 representative images (≥100 glands) of proximal stomach corpus were obtained from 3 mice at 20× magnification and then cell numbers of each lineage-specific marker including Ki-67, a proliferating cell marker in each gland were manually quantitated per gland using the Adobe Photoshop measurement tool. To measure the extent of macrophage infiltration after A939572 treatment, the area of F4/80-positive cells in 3 representative images per each mouse (n=3) was measured with ZEN 3.2 software (Zeiss) and divided by total area of each image at 20× magnification. All histological analyses were conducted blinded by two independent observers. The expression of SCD was determined by the intensity and percentage of epithelial cells expressing SCD were assessed for each core. Histo-scores (H-scores) were calculated by multiplying the intensity (0=negative; 1=weak; 2=moderate; 3=strong) and the percentage of positive epithelial cells (range=0-100), ranging from 0 to 300.

### RNA Extraction and Reverse-Transcription Quantitative PCR

Total RNA was extracted from mouse gastric organoids using the TRIzoI™ Reagent (15596026, Thermo Fisher Scientific) in accordance with the manufacturer's protocol. cDNA was synthesized from 1-μg of RNA using the iScript™ gDNA Clear cDNA Synthesis Kit (1725035, Bio-rad). Quantitative PCR was performed using the iTaq Universal SYBR Green Supermix (1725121, Bio-rad) according to the manufacturer's protocol on a CFX96™ Real-Time PCR Detection System. Expression data were normalized to Rplp0 mRNA levels. 2-ΔΔCt method was for relative quantification of gene expression (Table 3).

TABLE 3
Primer Sequences

| | | SEQ ID | | SEQ ID |
|---|---|---|---|---|
| Gene | Forward Primer (5′→3′) | NO: | Reverse Primer (5′→3′) | NO: |
| Acly | CCAGAGAAGGTTGCCCAAGTAT | 1 | TGTTGACGAACTCCATGGGAAT | 2 |
| Aldoa | ACACTGTACCAGAAGGCAGATG | 3 | TTCAAGTCATGGTCCCCATCAG | 4 |
| Cpt1a | AGATCCTGGATGACACCTCAGA | 5 | ACCACCTGTCGAAACATCTACC | 6 |
| Cpt2 | CAGAGAGGTGGCAAGGAATTCT | 7 | GTATGCAGGGTCCTGATAGAGC | 8 |
| Eno1 | GCCATGCAAGAGTTCATGATCC | 9 | AAGTCCAGGTCATACTTGCCAG | 10 |
| Fads1 | CATTGATCACGACCGGAATGTG | 11 | GAGTAAACAATGTCCGCGAAGG | 12 |
| Fads2 | ATCCCTTTCTACGGCATCTTGG | 13 | AGGTGGTGCTCAATCTGGAAAT | 14 |
| Fasn | TGGACTACTTTGTGGCCTTCTC | 15 | TGTGCTACAGCTTTCACCAGAT | 16 |
| Gapdh | AGGTCGGTGTGAACGGATTTG | 17 | GGGGTCGTTGATGGCAACA | 18 |
| Gpi1 | TACGGAAAGGTCTGCATCACAA | 19 | TAGTGGAGGAATCATGGGAGGT | 20 |
| Hk1 | GAAATGGGGCTGAGAAAGGAGA | 21 | TGTAGTCCAGGAAGTCGGAGAT | 22 |
| Ldha | GTGTGGAGTGGTGTGAATGTTG | 23 | TTAGAACTGCAGCTCCTTCTGG | 24 |
| Pdha1 | AGAGAGGATGGGCTCAAGTACT | 25 | TTAGCACAACCTCCTCTTCGTC | 26 |
| Pfkl1 | GCAGGAGCTTTGAGAACAACTG | 27 | ATGACAATGCAGAGTTCCTCGT | 28 |
| Pgam1 | ACCCCTTCTACAGCAACATCAG | 29 | TGAGAGACCCTCCAGATGCTTA | 30 |
| Pgk1 | TTGCAGACAAGATCCAGCTGAT | 31 | AATACCCCAACAGGACCATTCC | 32 |
| Pkm | TGGACAACGCTTACATGGAGAA | 33 | TGATGTTCTTGCCCTTCTCTCC | 34 |
| Rplp0 | GCTTCGTGTTCACCAAGGAGGA | 35 | GTCCTAGACCAGTGTTCTGAGC | 36 |
| Scd1 | CAAAGAGAAGGGCGGAAAACTG | 37 | CCAGGATATTCTCCCGGGATTG | 38 |
| Scd2 | GCAAGCTCTACACCTGTCTCTT | 39 | CAGTTTTCCGCCCTTCTCTTTG | 40 |
| Slc2a1 | AGTGTATCCTGTTGCCCTTCTG | 41 | GACCAGGGCCTACTTCAAAGAA | 42 |
| Slc16a1 | TCAGACCTCGGATCCAGTACTT | 43 | CTTCTCCTCCGCTTTCTGTTCT | 44 |
| Slc16a4 | CTCAGAAGTTCTCCAGTGCCAT | 45 | GGTATGAACCACCTCCCCATTT | 46 |
| Tpi | AGATGAGCTGATTGGCCAGAAA | 47 | AGCTTCTCGTGTACTTCCTGTG | 48 |

Imaging Mass Spectrometry Frozen stomach tissues from the Gif-rtTA;TetO-Cre;KrasG12D (GCK) mice were sectioned at 12-μm thickness and thaw-mounted onto indium-tin oxide (ITO) coated glass slides. MALDI matrix 9-aminoacridine (9AA) was spray-coated onto the MALDI target plates via an automatic sprayer (TM Sprayer; HTX Technologies). 9AA was made up as 5 mg/mL in 90% methanol, and four passes were used with a nozzle temperature of 85° C., a flow rate of 0.15 mL/min, 2-mm track spacing, and a stage velocity of 700 mm/min. Nitrogen was used as the nebulization gas and was set to 10-gauge pressure (psig). Images were acquired with a 15T Fourier transform ion cyclotron resonance mass spectrometer (FTICR MS, SolariX; Bruker Daltonics) equipped with an Apollo II dual ion source and Smartbeam II 2 kHz Nd:YAG laser that was frequency tripled to a 355-nm wavelength. Data were collected in the negative ion mode with the laser operating at 2 kHz. The pixel spacing was 50 μm (center-to-center distance) in both x and y dimensions. Data were collected from m/z 100-1400 with a resolving power of 190,000 at m/z 300. Tentative metabolite identifications were made by accurate mass, typically better than 1 ppm. MSiReader version 1.02 software was used for ion image visualization and data analysis. The pixel-wise calibration of metabolites was exported to imzML using Mmass software and summarized in Table 4.

TABLE 4
Average Peak Intensity for Imaging Mass Spectrometry

| Name | m/z | Untreated | PM | In-IM | HGD |
|---|---|---|---|---|---|
| FFA 16:1 | 253.217 | 9756.16 | 18108.9 | 49626.1 | 17176 |
| Palmitate 18:0 | 255.233 | 373761 | 109304 | 202350 | 201468 |
| Hexose phosphate | 259.023 | 162383 | 146361 | 174451 | 154276 |
| FFA 18:2 | 279.233 | 1489170 | 493332 | 594799 | 795719 |
| Oleate 18:1 | 281.248 | 1559780 | 1005230 | 1596270 | 1284240 |
| Stearate 18:0 | 283.264 | 1015320 | 860725 | 981340 | 1262200 |
| Arachidonate 20:4 | 303.232 | 655870 | 447462 | 341567 | 528229 |
| GSH | 306.077 | 256347 | 709989 | 329114 | 483748 |
| FFA 20:1 | 309.280 | 51583.5 | 67331.4 | 90717.3 | 130440 |
| DHA 22:6 | 327.233 | 149578 | 174613 | 108929 | 108389 |
| FFA 22:4 | 331.263 | 23083.5 | 33002.9 | 32396.2 | 69309.2 |
| Hexose | 338.988 | 209601 | 576000 | 579089 | 377272 |
| bisphosphate M-H | | | | | |
| Hexose | 376.944 | 138358 | 302221 | 243067 | 177655 |
| bisphosphate M2H | | | | | |
| LPA 16:0 | 409.236 | 336958 | 296481 | 173905 | 88299.5 |
| cPA 18:2 | 415.225 | 1624000 | 1420660 | 686143 | 331955 |
| cPA 18:0 | 419.256 | 1547940 | 4353570 | 2270050 | 2342270 |
| LPA 18:2 | 433.236 | 740490 | 239312 | 117241 | 78422.2 |
| LPA 18:1 | 435.252 | 278794 | 318926 | 295396 | 201808 |
| LPA 18:0 | 437.267 | 737519 | 877558 | 580450 | 509368 |
| Cholesterol sulfate | 465.304 | 1215940 | 1807040 | 1194020 | 1335760 |
| Taurocholate | 514.284 | 2362330 | 5569750 | 6732830 | 147681 |
| GSSG | 611.145 | 326371 | 1680580 | 505109 | 645980 |
| PA 36:4 | 695.465 | 662217 | 280371 | 125022 | 144585 |
| PA 36:3 | 697.482 | 413764 | 519489 | 230777 | 210762 |
| PA 38:5 | 721.481 | 98134.6 | 104298 | 46633.4 | 68447.8 |
| PI 38:4 | 885.552 | 4634410 | 3145430 | 1792580 | 3970510 |
| ST 24:1 | 888.627 | 3694.07 | 272540 | 89889.5 | 83415.1 |

### Organoid Culture and Drug Treatment

Mouse gastric organoids were established from corpus of untreated or Gif-rtTA; TetO-Cre; KrasG12D (GCK) mouse stomachs as described previously. The GCK mouse gastric organoids were cultured in ECM Gel (E1270, Sigma-Aldrich) or Cultrex® Reduced Growth Factor Basement Membrane Extract, Type R1 (3433-005-R1, R&D systems) with Mouse IntestiCult medium (06005, StemCell Technology) supplemented with 1% of penicillin/streptomycin (2441832, Gibco) in 48-well plates. The medium was replaced every 3 days and organoids were split every 3 to 5 days. Human gastric organoids were previously established from patients who underwent curative gastrectomy as described. The human gastric organoids were cultured in Corning™ Matrigel™ Membrane Matrix (356231, Thermo Fisher Scientific) with Human IntestiCult medium (06010, StemCell Technology) supplemented with 1% of penicillin/streptomycin, 0.2% of MycoZap (VZA-2031, Lonza) in a 48-well plate.

For metabolic enzyme inhibitor treatment, the media was switched to metabolic enzyme inhibitor containing media at one day after splitting the mouse gastric organoids and cultured for 3 days. Detailed information and final concentration of the inhibitors are provided in Table 5. For metabolite treatment, mouse gastric organoids were treated 100 nM A939572 with metabolites (cis-11-eicosenoic acid or cis-7,10,13,16-docosatetraenoic acid) at one day after splitting the mouse gastric organoids and cultured for 3 days. Detailed information and final concentration of the metabolites are provided in Table 5. The EVOS M7000 inverted microscope (Thermo Fisher Scientific) or JuLI™ stage, a Real-Time Cell History Recorder (NanoEntek) were used to capture phase contrast images of the organoids or monitor the growth and morphological changes of the organoids in real-time. Quantitative data analyses were performed using at least 3 wells of images at 4× magnification. For measurement of organoid growth, phase contrast images of organoids were captured over time and measured at the widest diameters using the ZEN 3.3 blue edition (Zeiss) measuring tool. For Calcein AM/Ethidium-1(EthD-1) staining, mouse gastric organoids were cultured for 1 day after splitting and transferred to microfluidic-based flowchips (Protein Fluidics) that can enable 3D cell-based assay. The media containing 100 nM A939572 with metabolites (cis-11-eicosenoic acid or cis-7,10,13,16-docosatetraenoic acid) was added to the top of the organoids and cultured for 3 days. Then, the organoids were stained with 2 μm Calcein AM/4 μm EthD-1 (L3224, Thermo Fisher Scientific) for 30 min at 37° C. Confocal imaging was performed on a Zeiss LSM 880 using a 20× magnification. To prepare Formalin-Fixed, Paraffin-Embedded (FFPE) organoid sections, whole mouse or human gastric organoids in Matrigel™ were fixed in 4% PFA for 30 min at RT and washed with 1×PBS twice. The organoids were embedded in Epredia™ HistoGel™ (HG-4000-012, Thermo Fisher Scientific) and processed according to a standard histological protocol for paraffin embedding.

TABLE 5
Inhibitors and Metabolites

| Inhibitor | Vendor | Cat# | Concentration | |
|---|---|---|---|---|
| 2-deoxy-D-glucose | MedChem Express | HY-13966 | 5 | mM |
| 6-Aminonicotinamide | Sigma-Aldrich | A68203 | 50 | μM |
| A939572 | MedChemExpress | HY-50709 | 100 nM for mouse; | |
| | | | 1 μM for human | |
| AP-III-a4 | MedChemExpress | HY-15858A | 10 | μM |
| BMS-303141 | MedChem Express | HY-16107 | 1 | μM |
| C75 | MedChemExpress | HY-12364 | 1 | μM |
| CBR-470-1 | MedChemExpress | HY-134205A | 10 | μM |
| ELOVL1-IN-1 | MedChem Express | HY-145122 | 1 | μM |
| ELOVL6-IN-2 | MedChem Express | HY-12146 | 1 | μM |
| FX-11 | MedChemExpress | HY-16214 | 10 | μM |
| GNE-140 racemate | MedChemExpress | HY-100742 | 10 | μM |
| GSK2837808A | MedChemExpress | HY-100681 | 10 | μM |
| Perhexiline maleate | Sigma-Aldrich | SML0120 | 10 | μM |
| SC-26196 | MedChemExpress | HY-107410 | 1 | μM |
| Shikonin | MedChem Express | HY-N0822 | 10 | μM |
| Telaglenastat | MedChemExpress | HY-12248 | 1 | μM |

| | | | |
|---|---|---|---|
| cis-7,10,13,16- | Sigma-Aldrich | D3659 | 1:100000 |
| docosatetraenoic acid | | | |
| cis-11-Eicosenoic acid | Sigma-Aldrich | E3635 | 1:100000 |

### Synthesis of NBD-Conjugated Metabolites

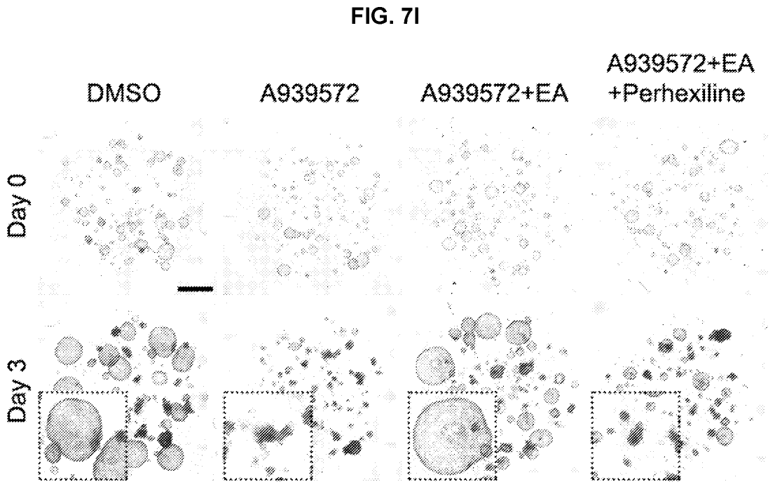

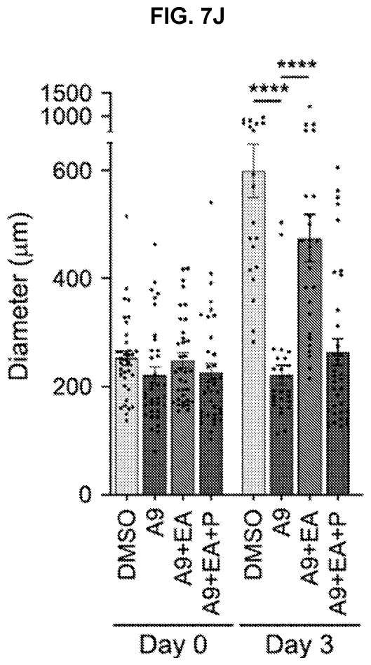

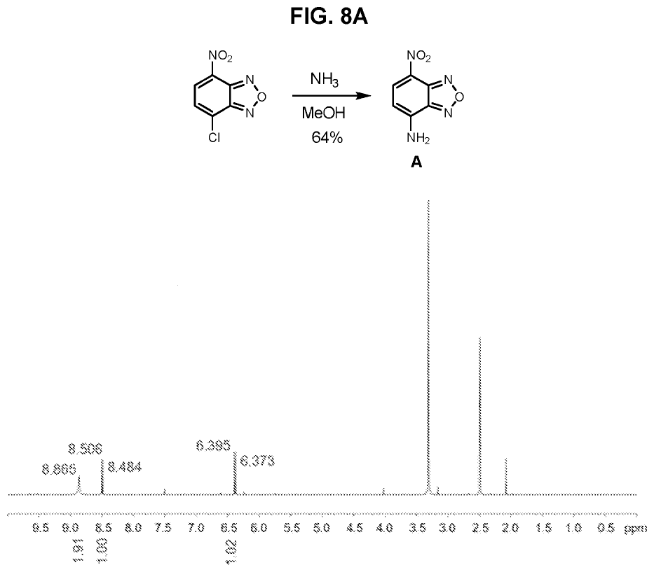

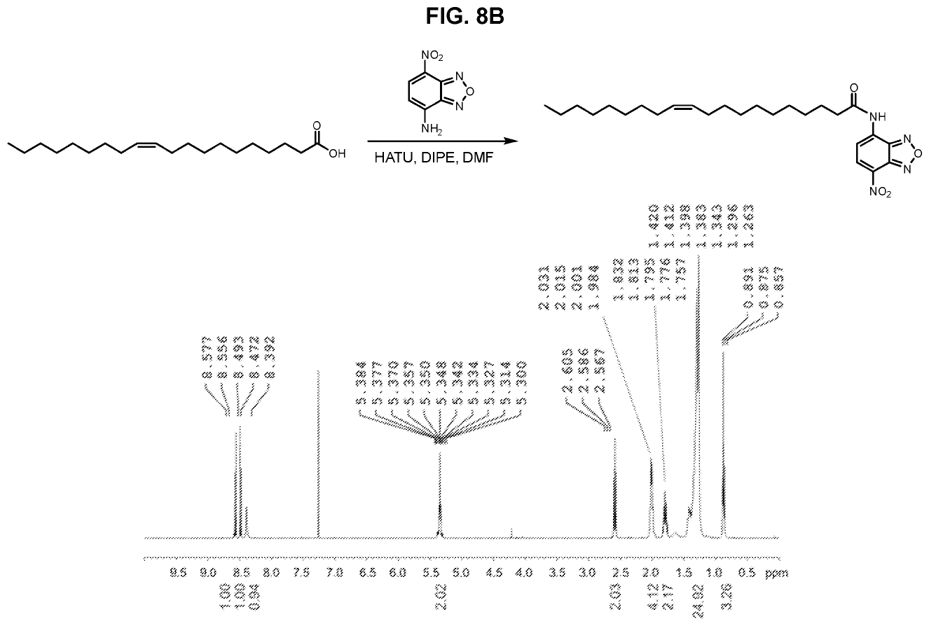

A schematic representation of the synthesis of NBD (nitrobenzoxadiazole;
nitrobenzofurazan)-eicosenoic acid (C20:1, n-9) is presented in FIG. 8. To prepare fluorescence-conjugated metabolites, solvents were obtained from either an MBraun MB-SPS solvent system or freshly distilled (tetrahydrofuran was distilled from sodium-benzophenone; toluene was distilled from calcium hydride and used immediately; dimethyl sulfoxide was distilled from calcium hydride and stored over 4 Å molecular sieves). Commercial reagents were used as received. Semi-preparative reverse phase HPLC was conducted on a Waters HPLC system using a Phenomenex Luna 5 μm C18(2) 100 Å Axia 250×10 mm column or preparative reverse phase HPLC (Gilson) using a Phenomenex Luna column (100 Å, 50×21.2 mm, 5 μm C18) with UV/Vis detection. Infrared spectra were obtained as thin films on NaCl plates using a Thermo Electron IR100 series instrument and are reported in terms of frequency of absorption (cm⁻¹). ¹H NMR spectra were recorded on Bruker 400, 500, or 600 MHz spectrometers and are reported relative to deuterated solvent signals. Data for ¹H NMR spectra are reported as follows: chemical shift (δ ppm), multiplicity (s=singlet, d=doublet, t=triplet, q=quartet, p=pentet, m=multiplet, br=broad, app=apparent), coupling constants (Hz), and integration. ¹³C NMR spectra were recorded on Bruker 100, 125, or 150 MHz spectrometers and are reported relative to deuterated solvent signals. LC/MS was conducted and recorded on an Agilent Technologies 6130 Quadrupole instrument.

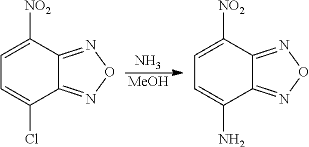

### Compound A (NBD; 7-nitrobenzo[c][1,2,5]oxadiazol-4-amine; nitrobenzoxadiazole; nitrobenzofurazan)

To a solution of 4-chloro-7-nitrobenzo[c][1,2,5]oxadiazole (1.0 g, 5.0 mmol) in MeOH (25 mL) was added ammonia solution (7.1 mL, 7 M in MeOH) at room temperature under argon atmosphere. The solvent was evaporated in vacuo, and then the crude product was purified by ISCO column chromatography eluting with 0 to 40% EtOAc in dichloromethane to provide the brown solid title Compound A (7-nitrobenzo[c][1,2,5]oxadiazol-4-amine) (0.58 g, 64% yield). ¹H NMR (400 MHz, MDSO-d₆) δ 8.87 (s, 2H, —NH₂), 8.49 (d, J=8.8 Hz 1H), 6.38 (d, J=8.8 Hz 1H); LCMS ESI-MS (m/z) calculated for C₆H₄N₄O₃[M+H]⁺: 181.03, measured 181.05.

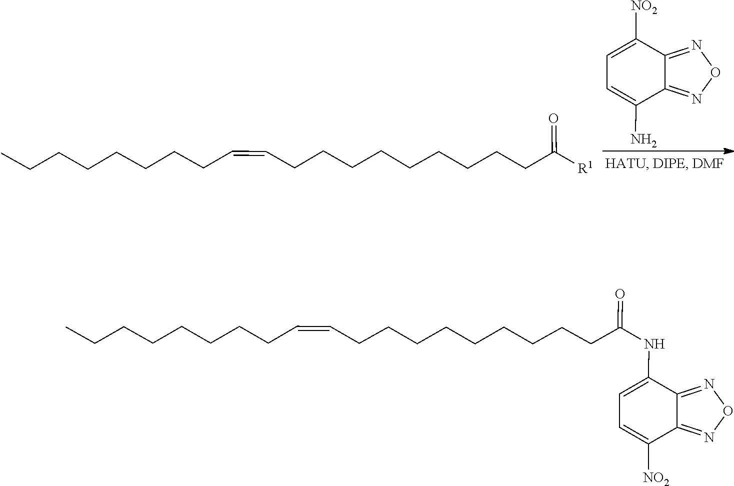

### NBD-cis-11-Eicosenoic acid (C20:1, n-9)

To a solution of cis-11-eicosenoic acid 1 (40 mg, 0.13 mmol) in DMF (1.6 mL) was added DIPEA (91 mL, 0.64 mmol), followed by addition of HBTU (99 mg, 0.26 mmol) at 0° C. The reaction mixture was stirred at 0° C. for 30 min, and then compound A (31 mg, 0.17 mmol) was added to reaction mixture at 0° C. The reaction mixture was stirred for 16 hr at room temperature, diluted with EtOAc (10 mL), washed with aqueous citric acid (1 M, 2×10 mL) and brine (10 mL). The combined organic phase was dried over MgSO₄, filtered, and concentrated in vacuo. The crude product was purified by ISCO column chromatography eluting with 0 to 25% EtOAc in hexane to provide the brown solid product (34 mg, 55% yield). ¹H NMR (400 MHz, CDCl₃) δ 8.56 (d, J=8.4 Hz, 1H), 8.48 (d, J=8.4 Hz, 1H), 8.39 (s, 1H, —NH), 5.38-5.30 (m, 2H), 2.58 (t, J=7.6 Hz, 2H), 2.03-1.98 (m, 4H), 1.83-1.76 (m, 2H), 1.42-1.26 (m, 24H), 0.87 (t, J=6.4 Hz, 3H).

### Two-Dimensional Monolayer Culture

Mouse gastric organoids were dissociated into single cells and dissociated cells (5,000 cells/well) were seeded on the collagen-coated 8-well chamber slide (ibidi) in Mouse IntestiCult medium (06005, StemCell Technology) for two days until the cells reach at 50% confluency. The cells were incubated with NBD (Compound A) or NBD-conjugated Eicosenoic acid over time and then stained with 100 nM MitoTracker™ Red CMXRos (M7512, Thermo Fisher Scientific) for 20 min at 37° C. The cells were washed with pre-warmed 1×PBS three times and counterstained with Hoechst for 5 min at RT. Confocal imaging was performed on a Zeiss LSM 880 using 63× magnification.

### Statistical Analyses

Statistical analyses were performed using GraphPad Prism (GraphPad Software, Inc). For 2-group comparisons, unpaired two-tailed Student's t test was performed to calculate P-values and to determine statistically significant differences. For multiple comparisons, One-way ANOVA followed by Tukey's post hoc test was used. Every experiment was repeated at least three times. The number of independent experimental replications, variation (mean±SD or mean±SEM) and statistical test (P-value) were reported in each corresponding figure legend.

### Example 2

### Kras Activation in Gastric Chief Cells Leads to the Sequential Development of Metaplasia and High-Grade Dysplasia

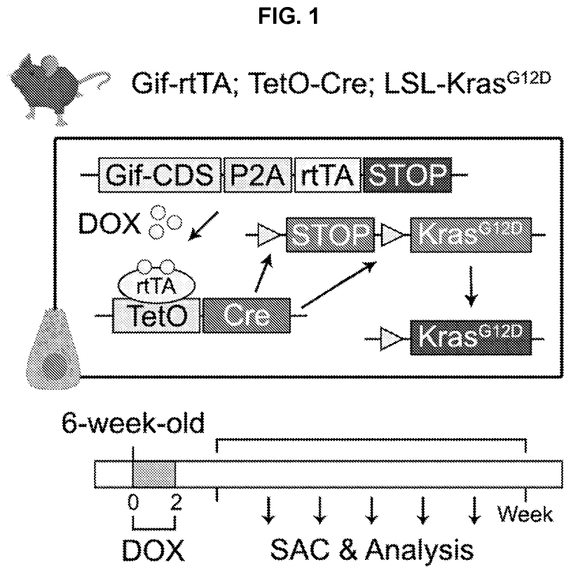

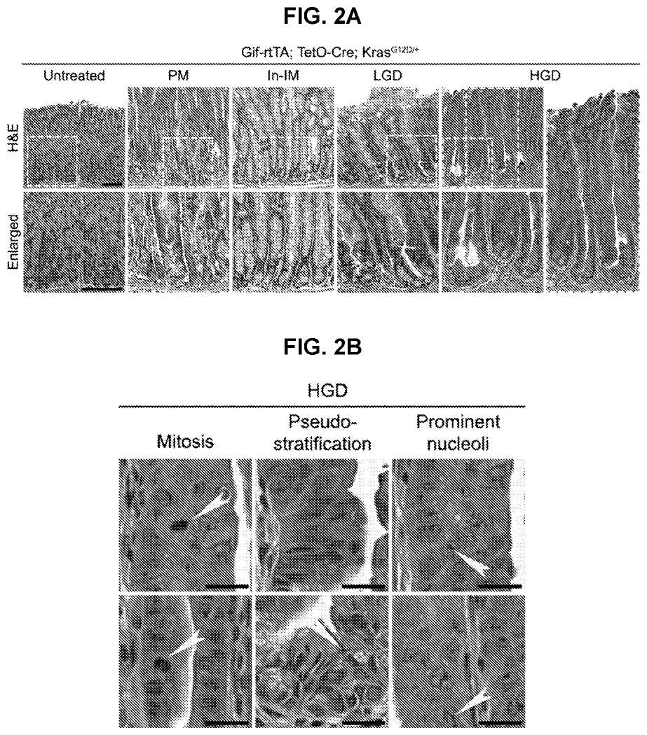

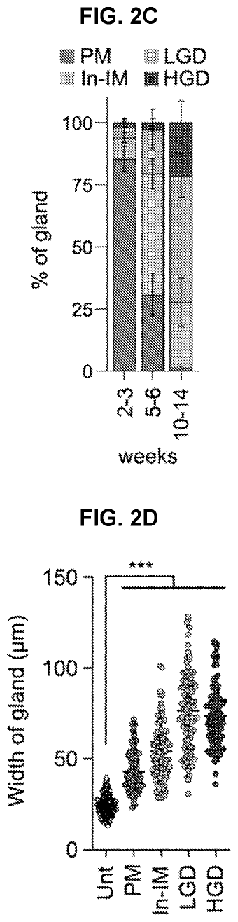

To determine whether the Kras activation only in gastric chief cells can lead to the entire process of gastric carcinogenesis, a novel transgenic mouse allele, Gif-rtTA;TetO-Cre; Kras^{G12D} (GCK), was generated by crossing the Gif-rtTA mice, a dox-inducible and chief cell-specific driver mouse allele, with the TetO-Cre and Kras^{G12D} alleles (FIG. 1). The GCK mice led to sequential development of metaplasia followed by low- and high-grade dysplasia within 10-14 weeks after doxycycline treatment (FIG. 2A). These mice developed pyloric metaplasia which consists of distinct cell types such as spasmolytic polypeptide expressing metaplasia (SPEM) cells, positive for CD44v9 and AQP5, at the base of glands and foveolar cells above the SPEM cell zone. The pyloric metaplasia gradually progressed to intestinal-type metaplastic and dysplastic glands. TFF3 expressed in the luminal cells of intestinal-type metaplastic glands. The TFF3 expression was significantly increased in the glands with SPEM cells at the base at 5-6 weeks, which is a typical gland composition of incomplete intestinal metaplasia (In-IM). These In-IM glands progressed to low-grade dysplasia (LGD) or even high-grade dysplasia (HGD) in about 25% of the mucosa at 10-14 weeks (FIG. 2A-C). The HGD recapitulated the structures of dysplastic glands and exhibited all the characteristics of dysplastic cells, including hyperchromatic nuclei, pseudo-stratification, and prominent nucleoli, commonly seen in human patients (FIG. 2B). While the number of TFF3-positive IM cells was dramatically decreased in the HGD, SPEM cells were still present at the base of HGD with a decreased co-expression of CD44v9 and AQP5. The LGD glands exhibited a transitioning cell zone in which cell lineage converts from metaplastic to dysplastic cells above the SPEM cell zone and confirmed by upregulation of the TROP2 expression, a key transition marker between metaplastic and dysplastic cells. Expression of CD133, a marker of dysplastic stem cells (DSCs) which contribute to adenocarcinoma development, was present in the apical membrane of cells in the transitioning cell zone in HGD. Moreover, CEACAM5, a dysplasia and gastric adenocarcinoma marker in human, was present only in the apical membrane of dysplastic cells and the expression was significantly increased in HGD. One distinct feature was that glands in Kras-induced stomachs continued to increase in width, especially between LGD and HGD stages (FIG. 2D). Also, the cell proliferation zone and level were expanded throughout the glands in HGD and aligned with a distribution of CEACAM5-positive dysplastic cells. CLDN3, a major structural molecule of tight junctions, was substantially decreased at the very base of glands in LGD and HGD, suggesting the loss of cell polarity and architectural changes in dysplastic glands by modifying tight junction structures. Therefore, these data demonstrate that Kras activation only in chief cells is sufficient to drive carcinogenic process to high-grade dysplasia with dynamic changes in cell lineage evolution as well as architectural changes in gland structures.

### Example 3

### Metabolic Reprogramming Turns on FA Metabolism During the Progression of Metaplasia to Dysplasia

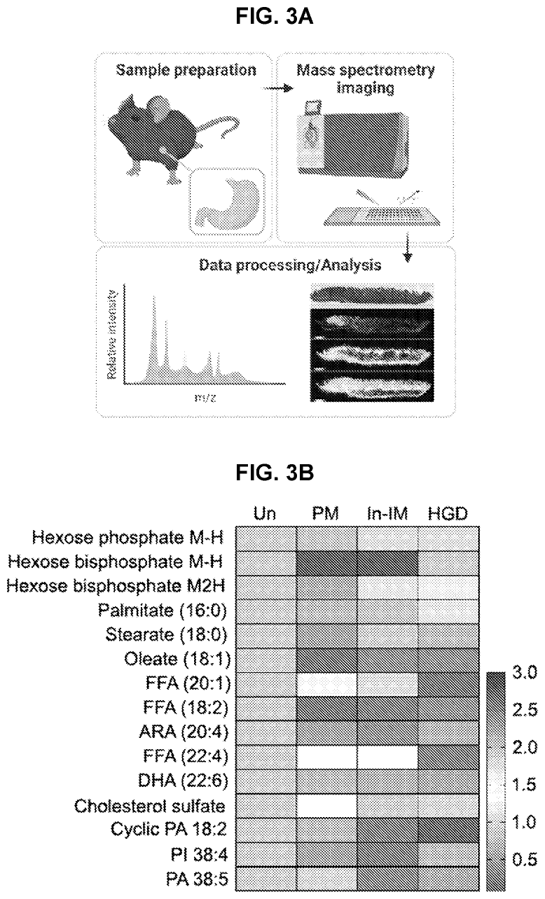

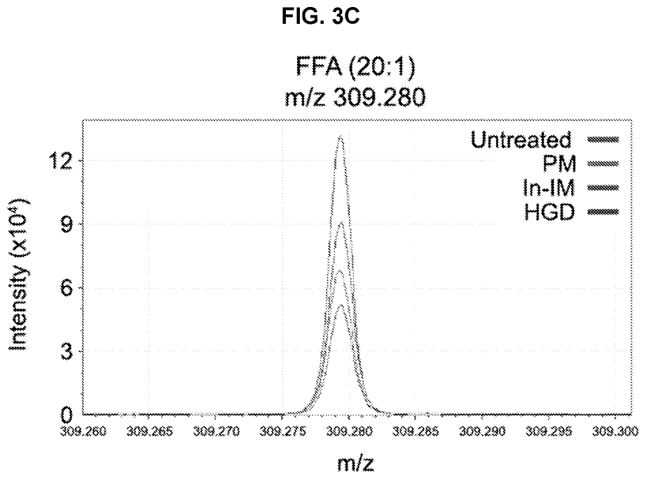

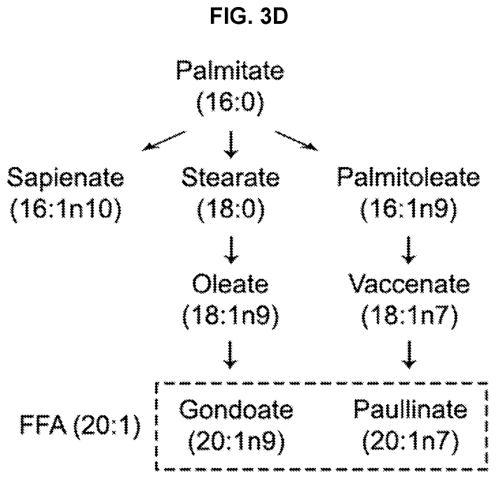

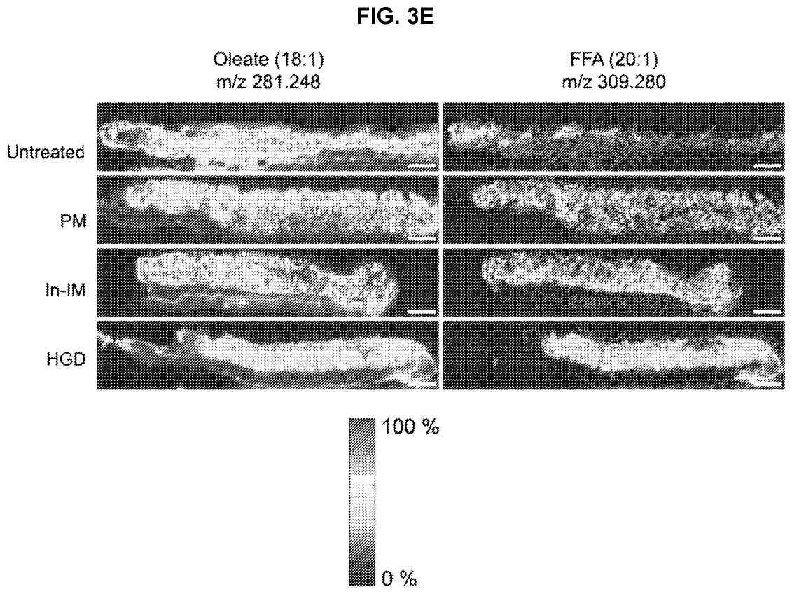

To identify metabolic pathways involved in the carcinogenic process, matrix-assisted laser desorption/ionization imaging mass spectrometry (MALDI-IMS) was performed using the GCK stomach tissues (FIG. 3A). A series of mass spectra (200-1400 m/z) for metabolites were collected over the tissue area. Key metabolites associated with major metabolic pathways, such as glycolysis, fatty acid (FA), phospholipid, glutathione metabolism were profiled and metabolites abundant in the metaplastic and/or dysplastic stages through in situ visualization of spatial distribution and quantitation were selected (FIG. 3B). Hexose bisphosphates, glycolytic pathway-associated metabolites, were first elevated in stomachs with pyloric metaplasia and gradually decreased during the progression to dysplasia (FIG. 3B). In contrast, long chain FAs, associated with FA metabolism, were differentially accumulated during progression. Palmitate is the most common saturated FA among long chain FAs in the human body. While the palmitate was abundant in normal stomachs, the level was significantly decreased in pyloric metaplasia and then increased in In-IM and HGD. In particular, one unique form of monounsaturated fatty acid (MUFA, FFA 20:1), which is an elongated form of FFA 18:1 by 2 carbon units (FIG. 3D), showed a gradual increase during the metaplasia progression and highly accumulated in HGD (FIG. 3C). Furthermore, the accumulation of the MUFA (FFA 20:1) was most prominent at the base of glands where cell lineage conversion is happening (FIG. 3E). It is important to note that oleate (18:1), a well-known form of MUFA produced from palmitate, was also abundant in the stomachs (FIG. 3E). However, there was no correlation between accumulation pattern of oleate and metaplasia progression. One polyunsaturated fatty acid (PUFA, FFA 22:4) also displayed a similar accumulation pattern to the MUFA (FFA 20:1). However, this PUFA (FFA 22:4) uses linoleate as a precursor which is obtained only from the diet. Therefore, these results revealed that a metabolic switch from glycolysis to fatty acid metabolism arises during the progression of metaplasia to dysplasia and produces a unique form of MUFA (FFA 20:1).

### Example 4

### SCD-Dependent FA Desaturation is Required for Dysplastic Cell Survival

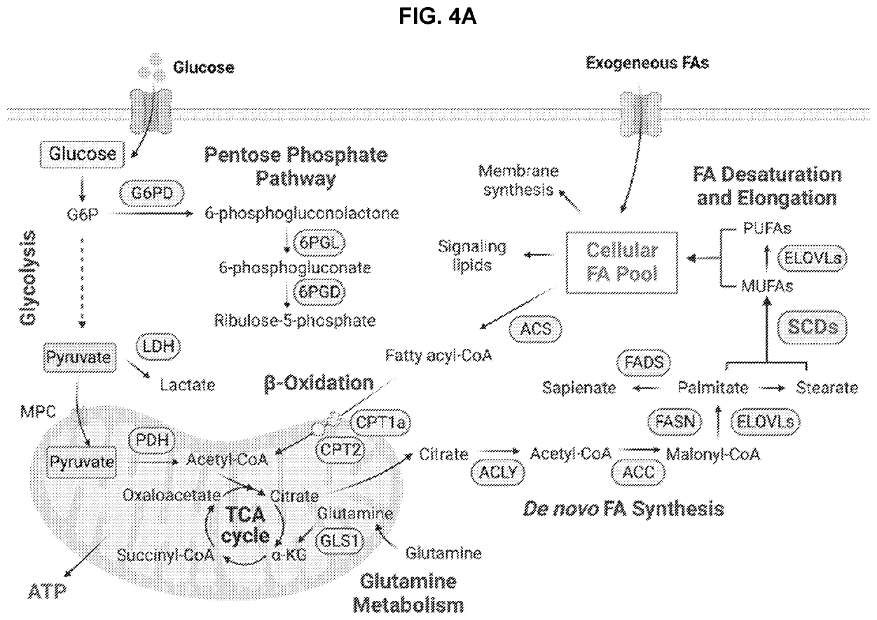

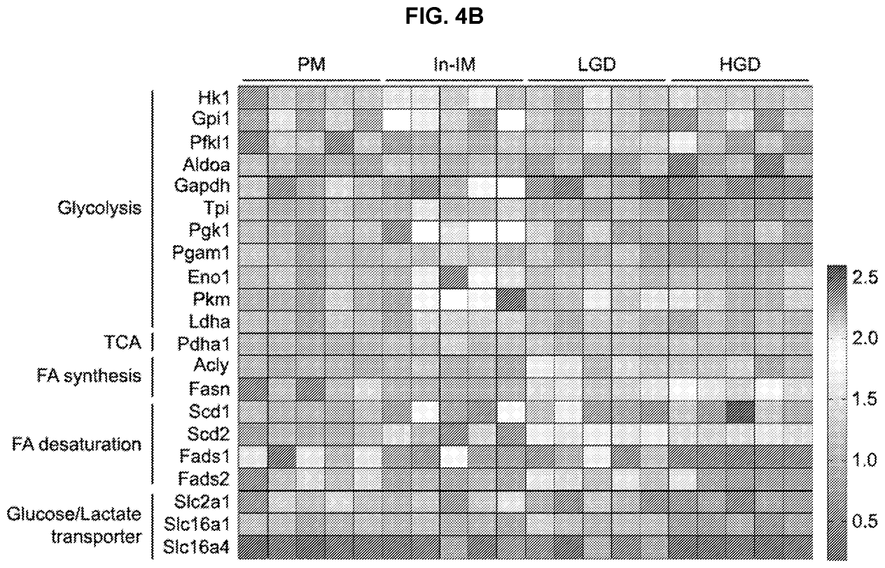

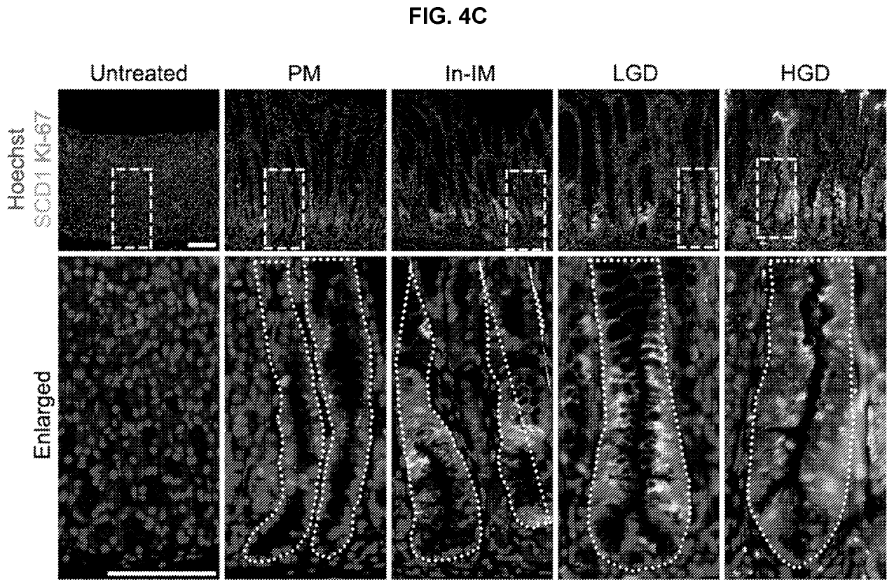

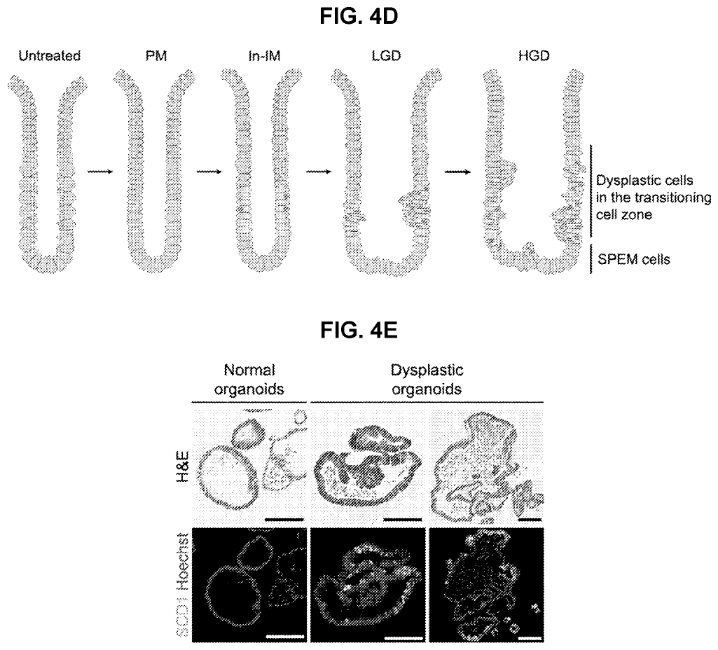

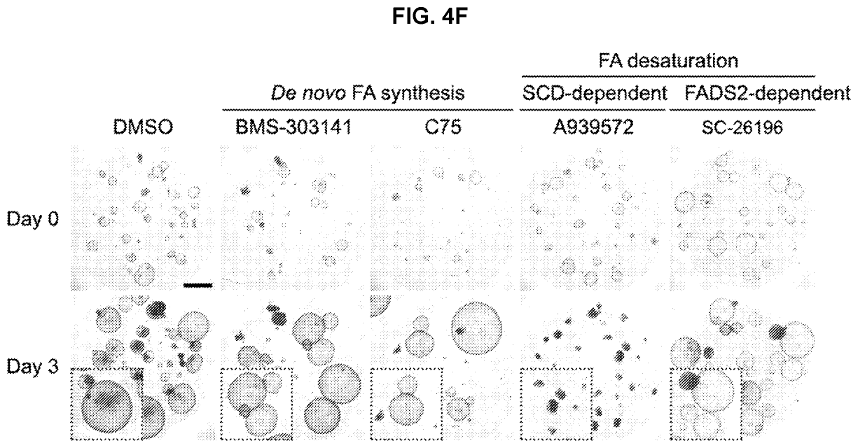

To determine the role of metabolic switch in dysplastic cells, expression levels of key metabolic enzymes, important for glycolysis or FA metabolism, were examined using gastric organoids established from each stage of GCK mice (FIG. 4A). Among the examined genes, expression level of glycolysis-associated enzymes was relatively high in In-IM organoids compared to pyloric metaplasia, LGD or HGD organoids. In contrast, FA desaturation pathway-associated enzymes, especially Scd1, were increased in LGD and HGD organoids (FIG. 4B). Stearoyl-CoA desaturase (SCD) is a rate-limiting enzyme which produces MUFAs. The SCD1-positive cells were often co-positive for Ki-67, a cell proliferation marker, and was prominently expressed in the transitioning cell zone both in LGD and HGD glands (FIG. 4C-D). The functional roles of metabolic pathways were assessed using key enzyme inhibitors in dysplastic organoids which highly expressed SCD1 (FIG. 4E). Dysplastic organoids were treated with small molecule inhibitors that target key steps in fundamental metabolic pathway including glycolysis and FA metabolism for 3 days. Inhibition of glycolysis did not affect either organoid viability or growth. Also, inhibition of pentose phosphate pathway and glutamine metabolism reduced organoid growth slightly but there was no effect on viability. Inhibition of de novo lipid synthesis and elongation, by targeting the ACLY, FASN, or ELOVL activity, had no effect on the survival of dysplastic organoids (FIG. 4F). However, blocking the FA desaturation by inhibiting the SCD activity, but not the FADS2 activity, resulted in 100% death in dysplastic organoids (FIG. 4F).

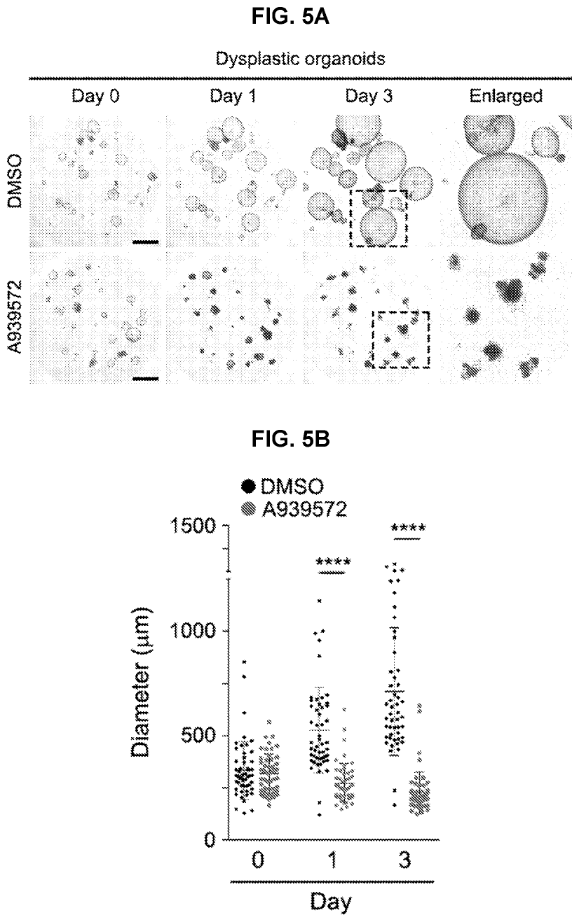

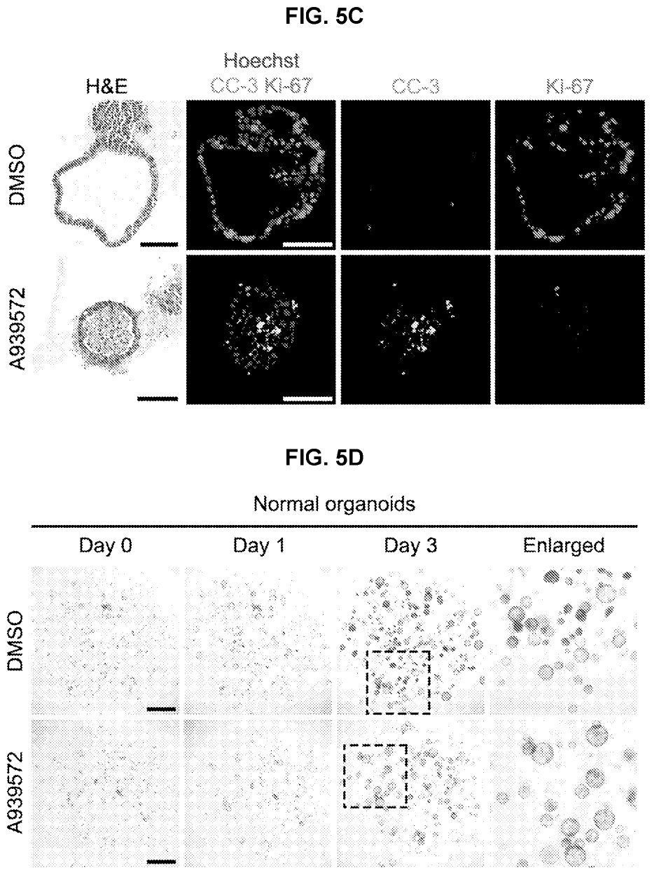

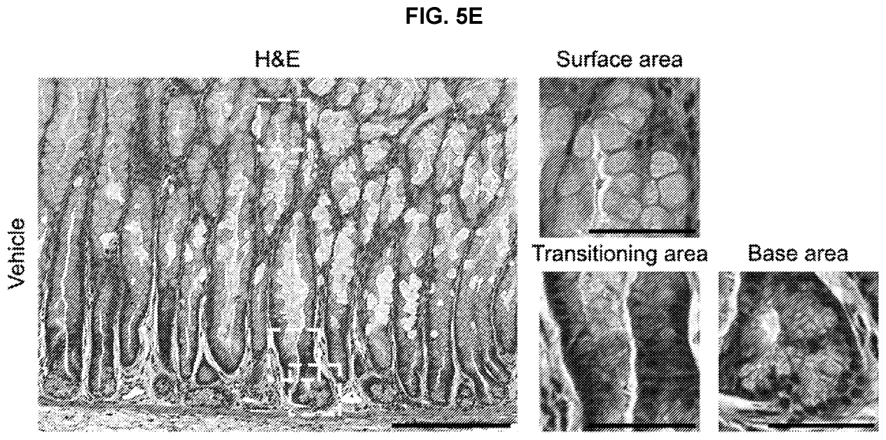

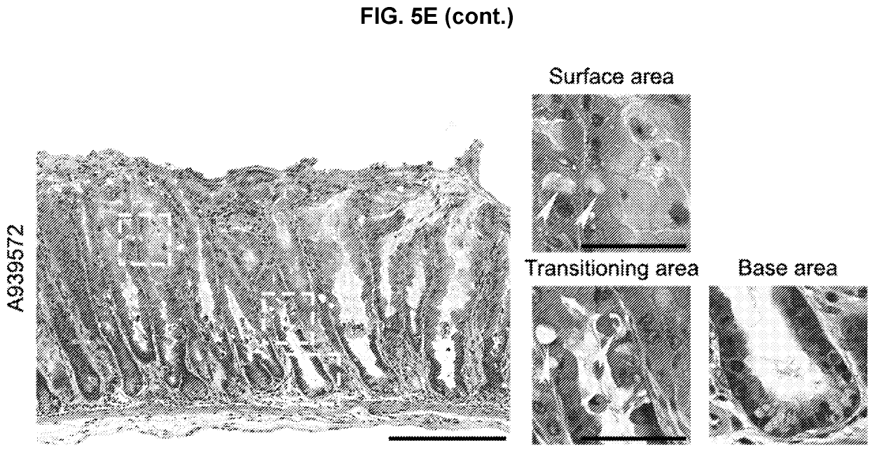

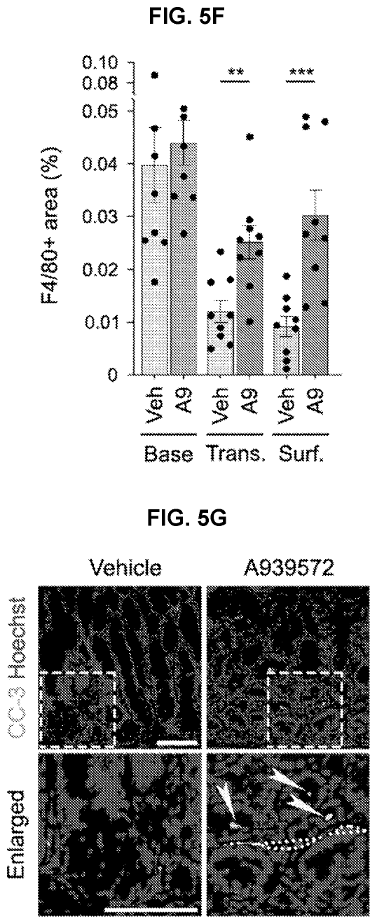

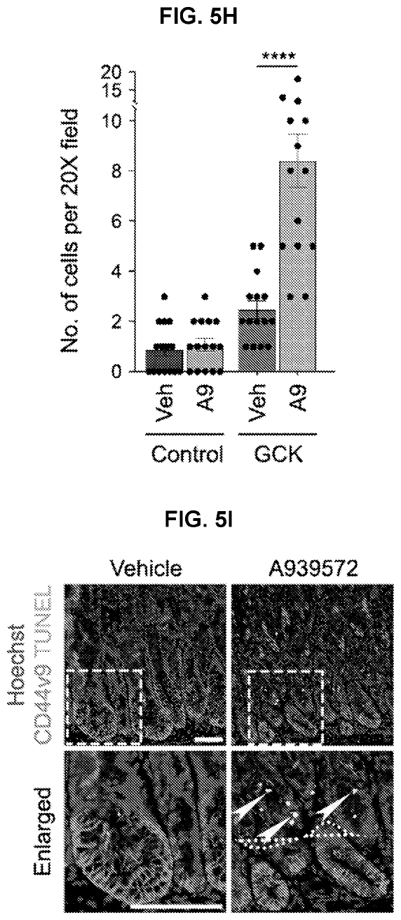

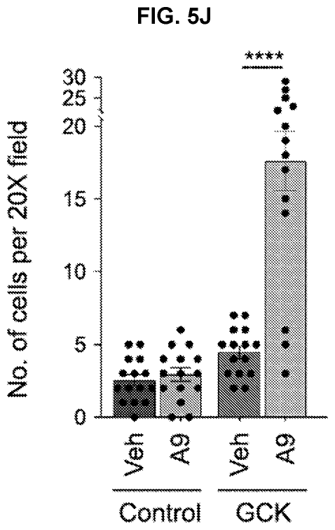

The importance of SCD-mediated FA desaturation was examined in dysplastic cells. SCD inhibition, using A939572, rapidly impeded dysplastic organoid growth and survival within 1 day (FIG. 5A-B). The organoids treated with A939572 showed an accumulation of cleaved caspase-3 positive apoptotic cells in the central lumen of dysplastic organoids and a decrease in cell proliferation (FIG. 5C). However, normal gastric organoids did not show any significant changes in response to the A939572 treatment (FIG. 5D). Furthermore, SCD inhibition specifically targeted dysplastic cells in vivo. In GCK mice treated with A939572 for 2 weeks at 6 weeks after doxycycline treatment, SCD inhibition resulted in a dramatic change in the gastric mucosa, without any effects in other organs (FIG. 5E). The GCK mice treated only with vehicle showed metaplasia progression to LGD which contains SPEM cells at the base, the transitioning cell zone, and IM cells in the surface area (FIG. 5E). In contrast, the GCK mice treated with A939572 displayed flattened mucosa and loss of histological characteristics of dysplastic cells in the transitioning and surface areas of glands. A significant increase in dead cells was observed within the transitioning cell zone, where the SCD is strongly expressed, and the cell death was confirmed by both cleaved caspase-3 and Terminal deoxynucleotidyl transferase dUTP nick end labeling (TUNEL) (FIG. 5G-J, yellow arrowheads). Additionally, F4/80⁺CD68⁻ macrophage infiltration was significantly increased in the transitioning and surface areas indicating an increase in phagocytic activity in the mucosa (FIG. 5F). F4/80⁺CD68⁺ macrophages, which promote metaplasia progression, was slightly increased only at the base of glands. Although significant degenerated changes were observed in gastric mucosa after the A939572 treatment, SPEM cells were still present at the gland base and not co-positive for cleaved caspase-3 or TUNEL signal (FIG. 5I). Therefore, these results indicate that SCD-dependent FA desaturation is required for dysplastic cell proliferation and survival.

### Example 5

SCD-Dependent FA Desaturation Produces an Unsaturated Long-Chain Fatty Acid that Fuels Dysplastic Cells

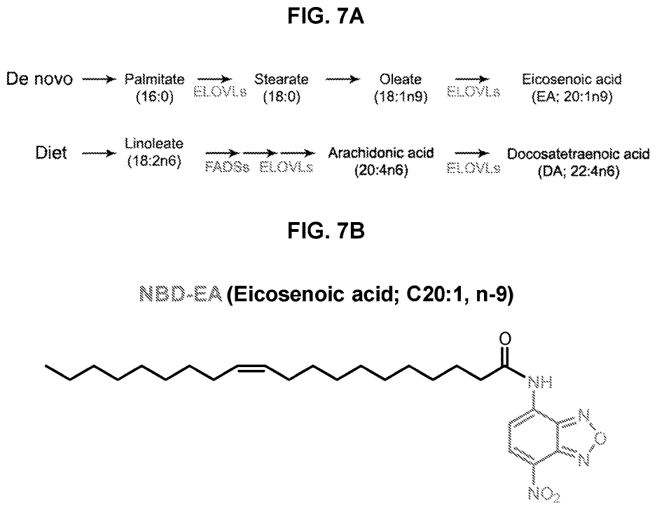

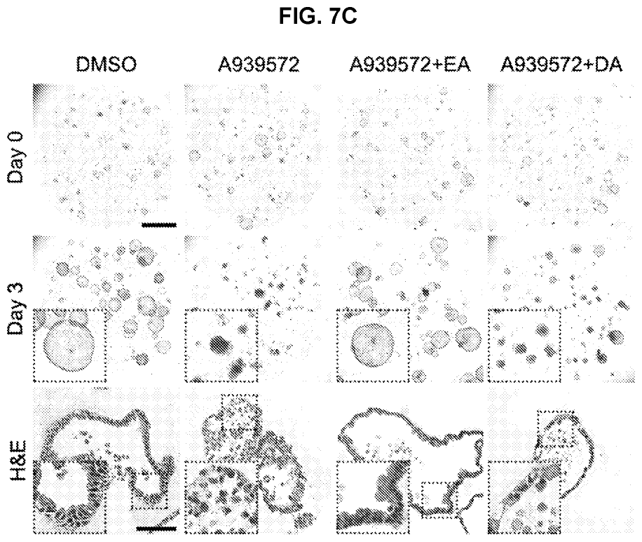

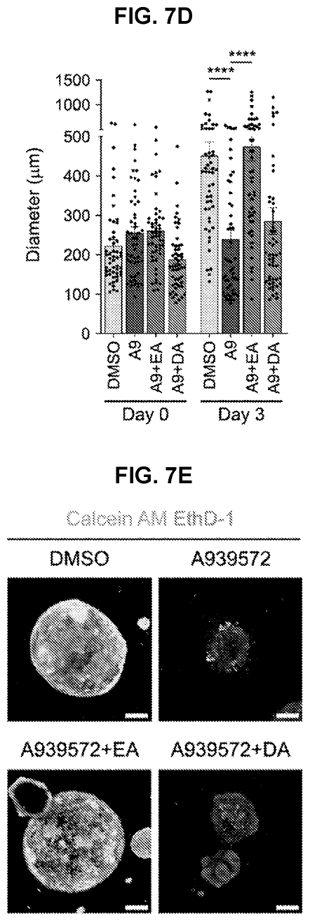

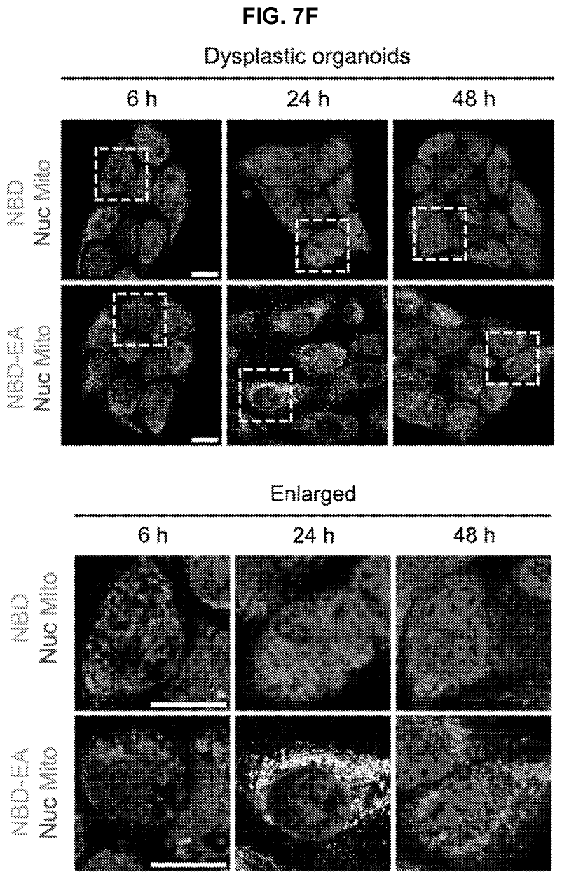

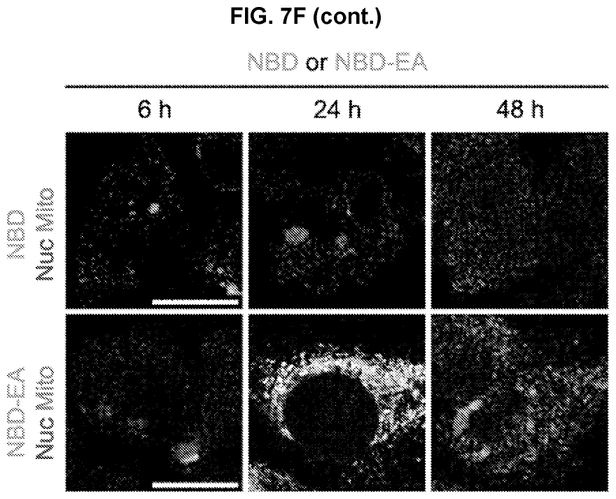

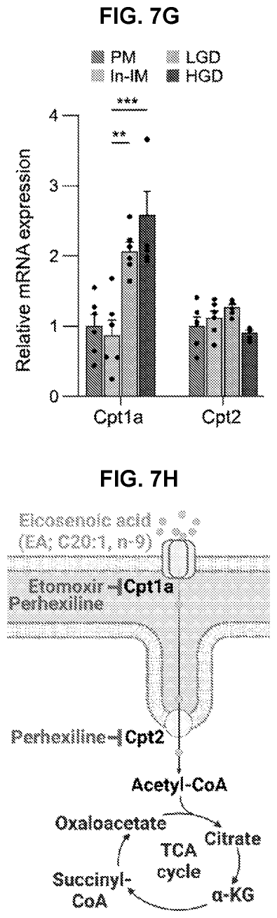

To address how and why dysplastic cells utilize the SCD-dependent FA desaturation, experiments examined the functions of two different unsaturated long-chain FAs, MUFA (FFA 20:1) and PUFA (FFA 22:4), which were abundant in dysplasia. Two metabolites, Eicosenoic acid (EA; 20:1n9, MUFA) and Docosatetraenoic acid (DA; 22:4n6, PUFA) (FIG. 7A) were selected based on the enhanced fragment ions detected from imaging mass spectrometry (FIG. 3E). Dysplastic organoids were co-treated with A939572, in combination with either EA or DA for 3 days. The organoids co-treated with A939572 and EA recovered from SCD inhibition and displayed a significant increase in size (FIG. 7C-E). Also, organoid structures were not disrupted and showed clear central lumens (FIG. 7C). However, the organoids co-treated with A939572 and DA did not show any differences compared to the organoids treated only with A939572. To determine the subcellular localization of EA in dysplastic cells, EA was conjugated with NBD, a green-fluorescent compound (NBD-EA) (FIG. 7B and FIG. 8A-B). NBD-EA was not detectable in 2-dimensional monolayer culture of dysplastic cells at 6 h after treatment, but the fluorescent signal was strongly accumulated in mitochondria at 24 h and weakly maintained until 48 h (FIG. 7F). Since the EA is imported into mitochondria, experiments further examined whether the EA undergoes FA oxidation which occurs in the mitochondrial aerobic metabolism and produces energy substrate for the TCA cycle. Carnitine palmitoyltransferase (CPT) shuttles long-chain FAs into the mitochondria and the expression of Cpt1a, an isoform of CPT1, was upregulated in dysplastic organoids (FIG. 7G). Inhibition of FA oxidation using perhexiline abolished rescue effects of EA in dysplastic organoids (FIG. 7H). The organoids co-treated with A939572, EA and perhexiline did not display any significant increase in organoid growth (FIG. 7I-J). Thus, these data suggest that dysplastic cells actively utilize EA as a major substrate for energy production in mitochondria.

### Example 6

### Metabolic Rewiring Pattern is Observed Across PreCancerous Lesions in GI Cancers

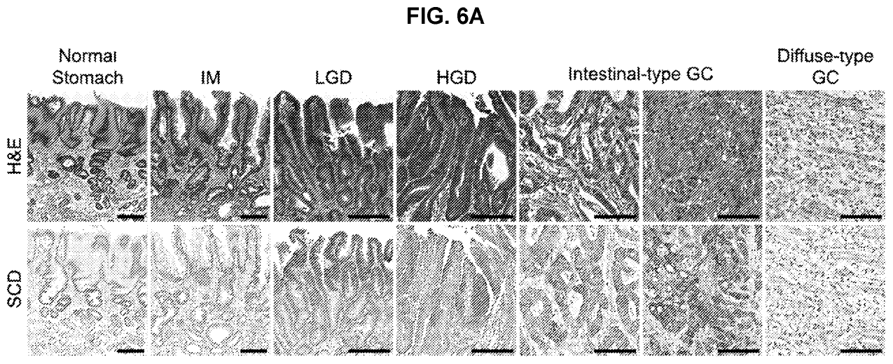

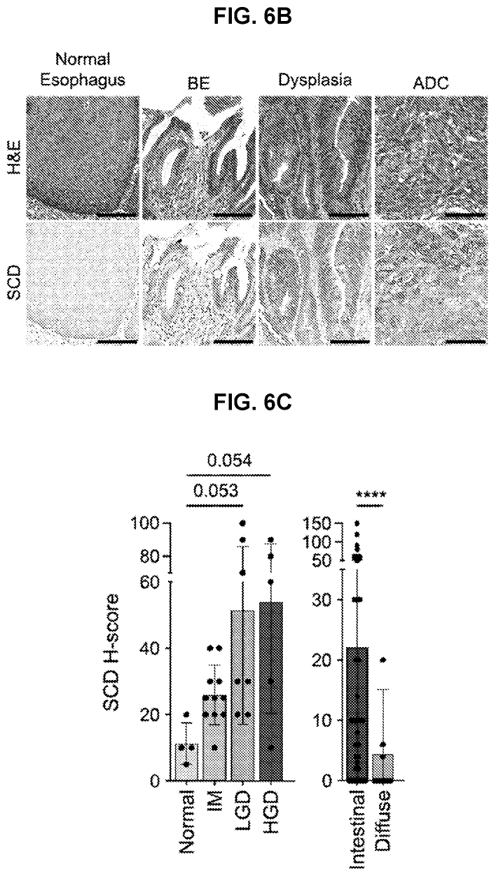

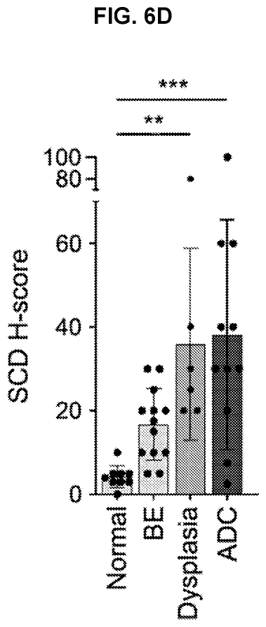

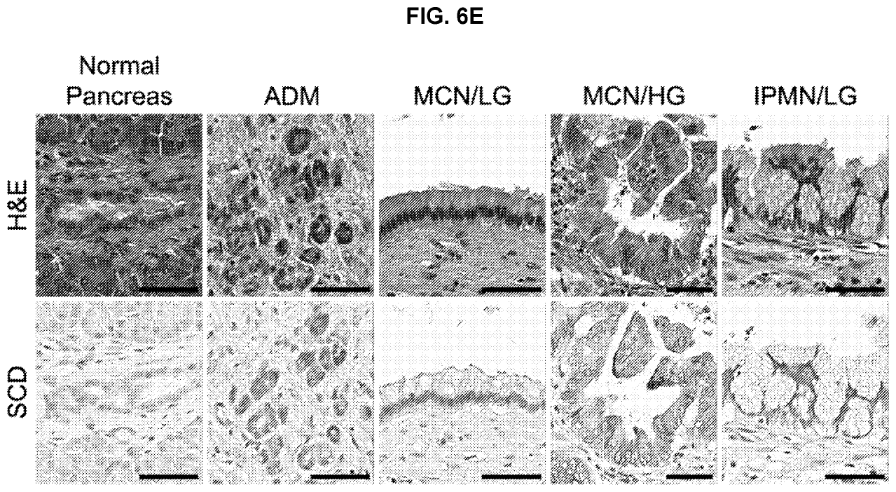

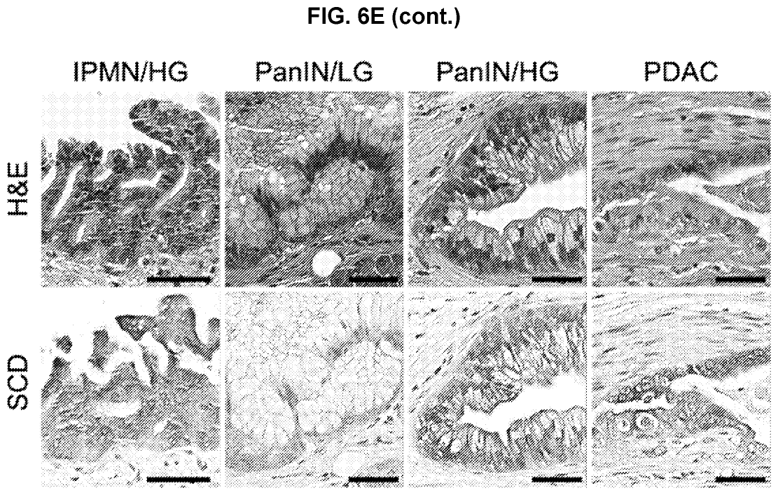

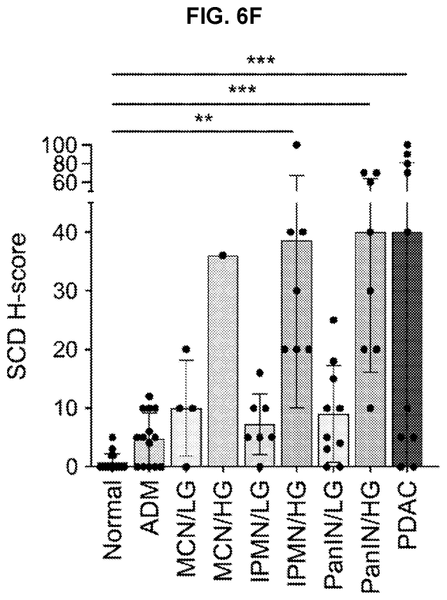

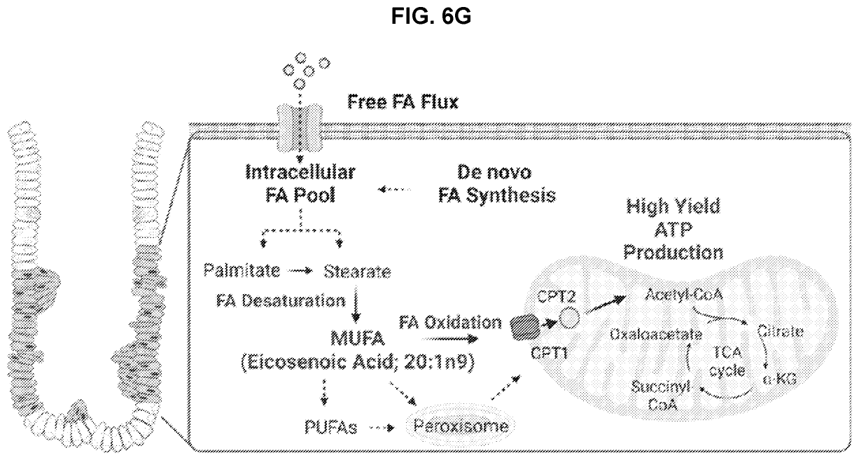

Since epithelial cells in other gastrointestinal (GI) tract organs also undergo a sequential progression of carcinogenesis, further studies determined whether SCD-dependent FA desaturation is also activated in the progression of precancerous stages to cancer across the human GI tract. SCD was immunostained as an indicator of the extent of SCD-dependent FA desaturation in multiple sets of GI patient tissue microarrays and sections composed of precancerous lesions and cancers in the stomach, esophagus, and pancreas (FIG. 6). In gastric cancer tissues, SCD expression was significantly increased in dysplastic lesions, both LGD and HGD, compared to matched pairs of normal and metaplastic lesions (FIG. 6A). The SCD was highly expressed in intestinal-type gastric cancer, which develops within a carcinogenic cascade, compared to diffuse-type gastric cancer (FIG. 6A, 6C). Esophageal adenocarcinoma develops from precancerous metaplasia, known as Barrett's esophagus, to dysplasia and adenocarcinoma and the SCD expression was significantly increased in esophageal dysplasia and adenocarcinoma (FIG. 6B, 6D). Lastly in pancreatic cancer, three predominant types including mucinous cystic neoplasia, intraductal papillary mucinous neoplasia, and pancreatic intraepithelial neoplasia are defined as precancerous lesions. SCD expression was elevated in high-grade dysplasia of all three types and adenocarcinoma, compared to normal or low-grade lesions (FIG. 6E-F). Therefore, these results indicate that SCD upregulation is a common feature observed during the metaplasia progression to dysplasia in GI tract carcinogenesis.

To assess whether the SCD-dependent FA desaturation is necessary for human dysplastic cell survival, 9 human gastric organoid lines, derived from patient samples with metaplasia or dysplasia, were treated with A939572. The organoid lines exhibited a broad spectrum of sensitivity to SCD inhibition. Organoid lines, which displayed dysplastic histology and expressed both SCD and Trop2, did not grow and died within 6 days after the treatment. However, the other group of organoids which expressed low level of SCD did not respond to the treatment. Also, those organoids displayed a single monolayer and were negative for Trop2, indicating metaplastic organoids. These results collectively suggest that SCD function is important for dysplastic cell survival and that SCD-dependent FA desaturation might be activated during the carcinogenic transition from precancerous metaplasia.

This study focused on cellular changes in dysplasia induced by Kras activation in zymogen-secreting chief cells in the stomach using the GCK mouse allele. Kras activation generally induces only precancerous metaplasia in other GI tract organs and several additional oncogenic gene activation or mutations are necessary for progression of the precancerous lesions to malignant stages. In contrast, the GCK mice developed high-grade dysplasia through precancerous metaplasia progression. Histological phenotypes in the GCK mouse stomachs faithfully recapitulated the structures of metaplastic or dysplastic glands seen in human patients. Continuous cell lineage conversion above the SPEM cell zone denoted a main event of carcinogenic transformation of precancerous cells and distinguished the transitioning cell zone in both low- and high-grade dysplasia. Also, an expansion of proliferating cell zone and changes in cell adhesion and tight junction molecules, such as CEACAM5 and CLDN3, provided architectural progression of metaplastic glands to dysplastic glands. These changes might lead to the loss of cell polarity and asymmetric cell division that are key cytological features of dysplasia.

Cancer cells often reprogram their metabolism, and the metabolic switch has pivotal roles for supporting hyperproliferation and growth in cancer development. While glycolytic metabolism is a well-known pathway upregulated in cancer cells, aberrantly activated lipid metabolism can fuels cancer cell proliferation. However, it remains undefined whether metabolic switch occurs during the carcinogenic process or can regulate cellular dynamics in dysplastic cells. The imaging mass spectrometry allowed analysis of various types of metabolites abundant in stomach mucosa with different carcinogenic stages. While glycolytic metabolism was first activated during metaplasia development, it switched to fatty acid metabolism during metaplasia progression. In particular, a long chain monounsaturated fatty acid, Eicosenoic acid (EA; 20:1n9, MUFA), was observed in the transitioning cell zone of dysplastic glands. FA desaturation can be regulated by different enzymes such as the stearoyl-CoA desaturases (SCDs), and the FA desaturases (FADSs) in mammals. SCD expression is increased in many types of cancers and can control stemness in cancer stem cells, cancer cell proliferation, and even acquired resistance to chemotherapy. SCD-dependent FA desaturation actively occurs in dysplastic cells and produces EA. Multiple types of precancerous lesions exist in human epithelial cell carcinogenesis and the terms, order, and mutational signatures are also different between organs. However, a distinct, but common, pattern of SCD upregulation was identified in dysplasia and adenocarcinoma across human GI tract organs. This may suggest that metaplastic cells do not require FA metabolic reprogramming, but the reprogramming occurs in dysplastic cells and cancer cells in GI carcinogenesis.

Cancer-associated alterations in lipid metabolism include increased lipogenesis and lipid uptake from extracellular microenvironment as well as enhanced lipid storage in cells. In particular, FA desaturation is known to support membrane biosynthesis or prevent both lipotoxicity from excessive saturated FAs or ferroptosis triggered by lipid peroxidation. However, targeting FA desaturation using A939572, an SCD inhibitor, in GCK mice specifically killed dysplastic cells. Also, both mouse and human dysplastic organoids died within several days after the A939572 treatment. Chasing subcellular localization of EA using a synthesized fluorescence-conjugated EA revealed that EA can be translocated into the mitochondria and fuel dysplastic cell survival and proliferation through fatty acid oxidation. These results therefore indicate that metabolism reprogramming in dysplastic cells is required to meet their high energy demand for hyperproliferation and growth. Also, the EA production might be a hallmark of the metabolic switch in the dysplastic cells.

It is important to note that the inhibition of aerobic glycolysis (Warburg effect), which is the most well-known metabolic pathway in cancer cells, did not affect the dysplastic cell viability or growth. This supports a unique metabolic feature of dysplastic cell behavior, which utilizes FAs as a major energy source, rather than glucose. Although mammalian cells can use two major sources of free FAs through either de novo lipid synthesis or exogenous free FAs, it is not yet clear how the dysplastic cells generate the EA. The rescue experiment with the EA suggests that dysplastic cells can survive by exogenous uptake of free FAs. However, further studies are needed to evaluate the mechanisms of free FA influx into the dysplastic cells and its relative contributions to generate long chain FAs. Also, ELOVLs (Elongation of very long chain fatty acids protein) are potential candidates that can add substrates with carbon chains up to 20 in long chain FAs. Nevertheless, no significant effects on viability or growth in dysplastic organoids were observed after the inhibition of ELOVL1 or 6. Since very little is known about ELOVL functions underlying FA elongation and only limited ELOVL inhibitors have been developed, further studies will be needed to elucidate specific functions of FA elongation in dysplastic cells.

This study confirms that Kras activation in chief cells is sufficient for the full process of carcinogenesis to high-grade dysplasia in the stomach. Unlike cancer cells, where display a diverse cell population with distinct molecular and metabolic signatures, characteristics of dysplastic cells are relatively simple. Also, SCD upregulation in dysplastic cells drives FA metabolic reprogramming and produces an energy substrate, Eicosenoic acid (EA; 20:1n9, MUFA), through SCD1-dependent FA desaturation. Therefore, SCD-dependent FA desaturation is an essential element in onco-fatty acid metabolism for high energy demand required for dysplastic cell hyperproliferation and survival in epithelial carcinogenesis.

### Example 7

### Synthesis of Nitrobenzoselenadiazole-Conjugated Metabolites

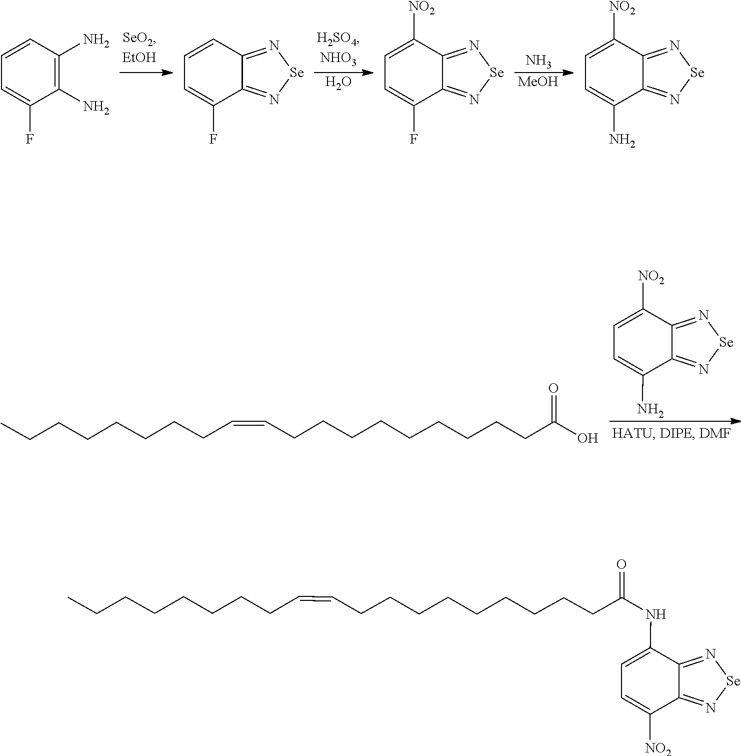

### Nitrobenzoselenadiazole-cis-11-Eicosenoic acid (C20:1, n-9)

Cis-11-eicosenoic acid was also conjugated to the fluorophore, nitrobenzoselenadiazole, which is a red fluorescent label described in Benson et al., Angew Chem Int Ed Engl., 131(21): 6985-6989 (2019). The fluorescent eicosenoic acid conjugate compound was synthesized as described below.

To a solution of cis-11-eicosenoic acid 1 (40 mg, 0.13 mmol) in DMF (1.6 mL) was added DIPEA (91 mL, 0.64 mmol), followed by addition of HBTU (99 mg, 0.26 mmol) at 0° C. The reaction mixture was stirred at 0° C. for 30 min, and then nitrobenzoselenadiazole (31 mg, 0.17 mmol) was added to reaction mixture at 0° C. The reaction mixture was stirred for 16 hr at room temperature, diluted with EtOAc (10 mL), washed with aqueous citric acid (1 M, 2×10 mL) and brine (10 mL). The combined organic phase was dried over MgSO₄, filtered, and concentrated in vacuo. The crude product was purified by ISCO column chromatography eluting with 0 to 25% EtOAc in hexane to provide the brown solid product.

### Example 8

### Metabolite Drug Conjugates (MDCs) for Inhibiting Dysplasia

Antibody drug conjugates (ADCs) have been actively developed for targeted therapeutics in recent years. Cytotoxins are commonly attached on the monoclonal antibody and negatively control cell replication or growth. However, many ADCs have also failed during clinical development due to issues with drug specificity and cytotoxic activities. Metabolite drug conjugates (MDCs) utilizing cis-11-eicosenoic acid provide an alternative targeted delivery platform.

Here, cis-11-eicosenoic acid was conjugated with toxins to inhibit dysplasia. This specific eicosenoic acid metabolite was found to be produced only in dysplastic cells, which are considered cancer-initiating cells. Patients with dysplasia in gastrointestinal organs have high risks for cancer development.

Metabolites such as cis-11-eicosenoic acid are small molecules which are used as intermediates of metabolic pathways or produced from the metabolic reaction. Various cytotoxins, including agents such as mertansine (DM1), taxol, calicheamicin, monomethyl auristatin E (MMAE), deruxtecan, SN-38, Pseudomonas exotoxin, diphtheria toxin, or Yttrium-90, can be conjugated to these metabolites. The small size and specificity of cis-11-eicosenoic acid is beneficial for drug conjugate and intracellular drug delivery. Therefore, some key benefits of the disclosed MDCs include that the cis-11-eicosenoic acid has cell-type specificity, is non-immunogenic, has low toxicity, and is easily internalized by epithelial cells.

Cis-11-eicosenoic acid was conjugated to the therapeutic toxin agent, mertansine (DM1, [(1S,2R,3S,5S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10,12,14(26),16,18-pentaen-6-yl](2S)-2-[methyl(3-sulfanylpropanoyl)amino]propanoate), as described below.

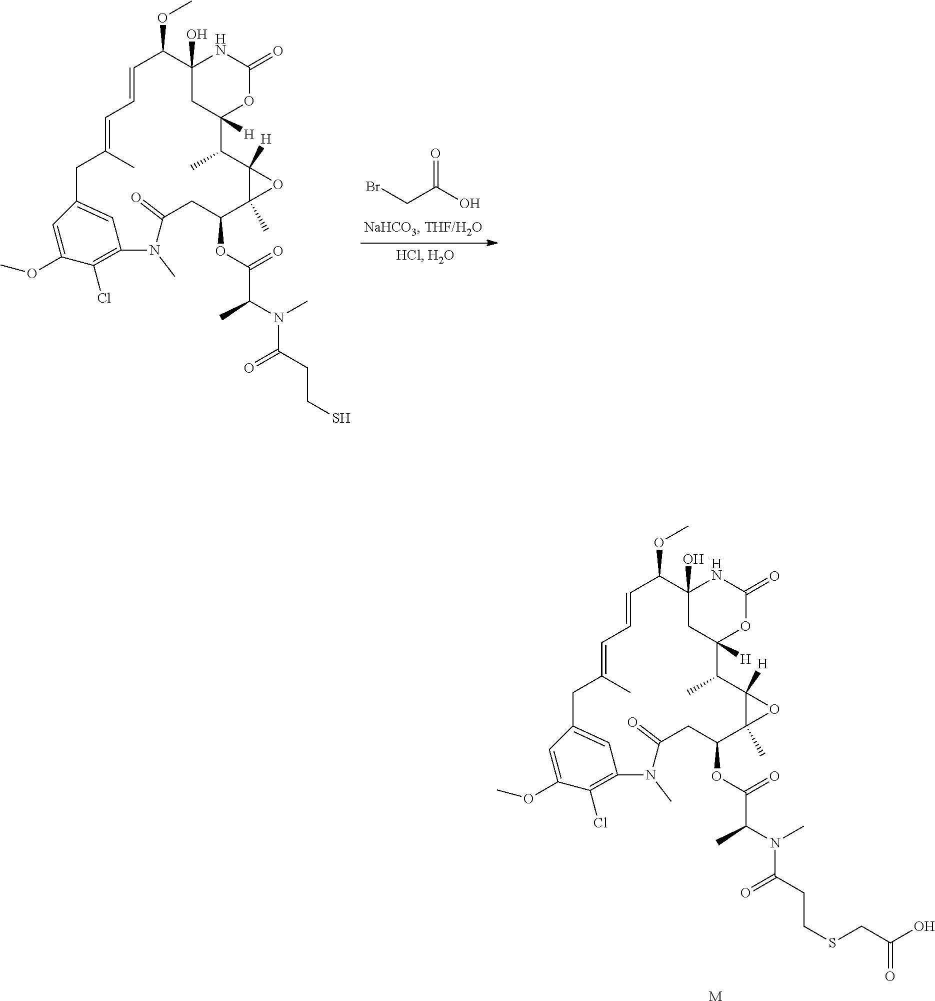

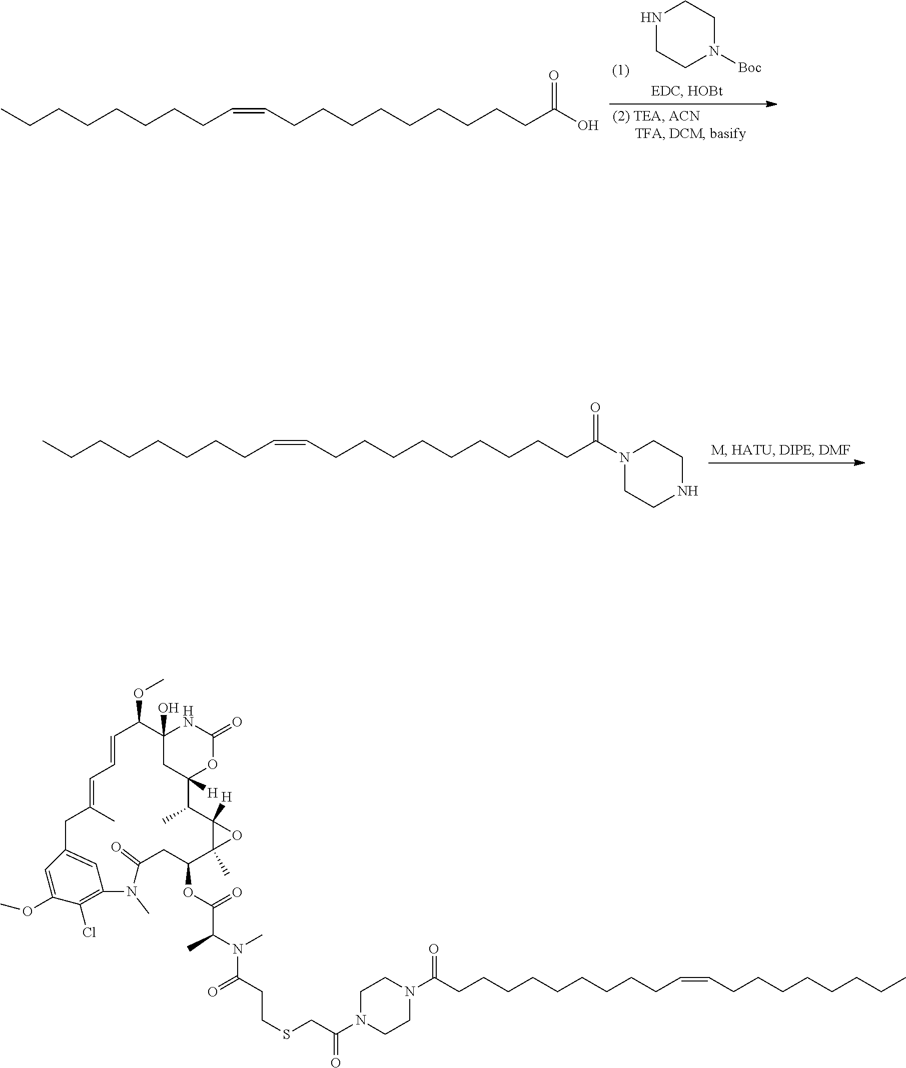 [SMILES: CC(C)(C)OC(=O)N1CCNCC1 ; CCCCCCCC/C=C\CCCCCCCCCC(=O)O ; CCCCCCCC/C=C\CCCCCCCCCC(=O)N1CCNCC1 ; CCCCCCCC/C=C\CCCCCCCCCC(=O)N1CCN(C(=O)CSCCC(=O)N(C)[C@@H](C)C(=O)O[C@H]2CC(=O)N(C)c3cc(cc(OC)c3Cl)C/C(C)=C/C=C/[C@@H](OC)[C@@]3(O)C[C@H](OC(=O)N3)[C@@H](C)[C@@H]3O[C@@]23C)CC1]

### (Z)-1-(Piperazin-1-yl)icos-11-en-1-one

### tert-Butyl (Z)-4-(icos-11-enoyl)piperazine-1-carboxylate

DECI (15.0 mg, 0.097 mmol), HOBt (17.5 mg, 0.130 mmol) and Et₃N (17 μL, 0.194 mmol) were added to a solution of (Z)-icos-11-enoic acid (2.00 mg, 0.064 mmol) in MeCN (2 mL) and stirred at room temperature for 45 min. tert-butyl piperazine-1-carboxylate (12.0 mg, 0.064 mmol) was added and stirred at 35° C. for 22 hr. The reaction was quenched with water and extracted with CH₂Cl₂ (3×5 mL). The combined organic layers were dried (MgSO₄), filtered, and concentrated. The residue was purified by silica gel chromatography with a Teledyne ISCO Combi-Flash eluting with 0 to 35% EtOAc in Hexanes to provide the desired product (26.0 mg, 84% yield). ¹H NMR (400 MHz, CDCl₃): δ 5.35-5.33 (m, 2H), 3.58 (t, J=5.3 Hz, 2H), 3.43 (s, 3H), 3.41-3.38 (m, 2H), 2.34-2.30 (m, 2H), 2.01 (quart. J=5.9 Hz, 4H), 1.67-1.58 (m, 4H), 1.48 (s, 9H), 1.35-1.25 (m, 23H), 0.88 (t, J=7.1 Hz, 3H); LC/MS 1.454 min, 479.0 (M+H).

### (Z)-1-(Piperazin-1-yl)icos-11-en-1-one

TFA (200 μL, 0.261 mmol) was added to a solution of tert-butyl (Z)-4-(icos-11-enoyl)piperazine-1-carboxylate (26.0 mg, 0.054 mmol) in CH₂Cl₂ (0.5 mL) and stirred at room temperature for 1 hr. The reaction mixture was concentrated under reduced pressure. The residue was dissolved in CH₂Cl₂ and treated with sat. aq. NaHCO₃ solution. The mixture was extracted with CH₂Cl₂ (3×5 mL). The combined organic layers were dried (MgSO₄), filtered, and concentrated to provide the desired product (20.0 mg, quant. yield). The crude compound was used for the next step without further purification. ¹H NMR (400 MHz, CDCl₃): δ 5.38-5.30 (m, 2H), 3.61-3.59 (m, 2H), 3.46-3.44 (m, 2H), 2.88-2.83 (m, 4H), 2.32-2.29 (m, 2H), 2.03-1.89 (m, 4H), 1.82-1.75 (m, 6H), 1.62 1 (quint. J=7.8 Hz, 2H), 1.35-1.27 (m, 19H), 0.88 (t, J=7.0 Hz, 3H); LC/MS 0.971 min, 379.1 (M+H).

### 2-((3-(((S)-1-(((1⁴S,1⁶S,3²S,3³R,2R,4S,10E,12E,14R)-8⁶-chloro-1⁴-hydroxy-8⁵,1⁴-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-1-oxopropan-2-yl)(methyl)amino)-3-oxopropyl)thio)acetic acid

Bromoacetic acid (14.3 mg, 0.103 mmol) and sat. aq. NaHCO₃ solution (270 μL) were added to a solution of (1⁴S,1⁶S,3²S,3³R,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(3-mercaptopropanoyl)-N-methyl-L-alaninate (10.0 mg, 0.014 mmol) and stirred at room temperature for 24 hr. The reaction was treated with acetic acid and extracted with CH₂Cl₂ (3×5 mL). The combined organic layers were dried (MgSO₄), filtered and concentrated. The residue was purified by silica gel chromatography with a Teledyne ISCO Combi-Flash eluting with 0 to 40% MeOH in CH₂Cl₂ to provide the desired product (5.2 mg, 49% yield). LC/MS 0.924 min, 777.6 (M-18).

### (1⁴S,1⁶S,3²S,3³R,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl N-(3-((2-(4-((Z)-icos-11-enoyl)piperazin-1-yl)-2-oxoethyl)thio)propanoyl)-N-methyl-L-alaninate

HATU (13.1 mg, 0.034 mmol) and N-methylmorpholine (3.8 μL, 0.035 mmol) were added to a solution of 2-((3-(((S)-1-(((1⁴S,1⁶S,3²S,3³R,2R,4S,10E,12E,14R)-8⁶-chloro-1⁴-hydroxy-8⁵,14-dimethoxy-3³,2,7,10-tetramethyl-1²,6-dioxo-7-aza-1(6,4)-oxazinana-3(2,3)-oxirana-8(1,3)-benzenacyclotetradecaphane-10,12-dien-4-yl)oxy)-1-oxopropan-2-yl)(methyl)amino)-3-oxopropyl)thio)acetic acid (13.7 mg, 0.017 mmol) in DMF (1.0 mL). After 5 min, (Z)-1-(piperazin-1-yl)icos-11-en-1-one (10.0 mg, 0.026 mmol) in DMF (1.0 mL) was added and stirred at room temperature for 5 min. The reaction was treated with water and extracted with EtOAc (3×5 mL). The combined organic layers were washed with water (6×), dried (MgSO₄), filtered, and concentrated. The residue was purified by silica gel chromatography with a Teledyne ISCO Combi-Flash eluting with 0 to 10% MeOH in CH₂Cl₂ to provide the desired product (13.5 mg, 68% yield); LC/MS 1.288 min, 1137.6 (M-18).

### Example 9

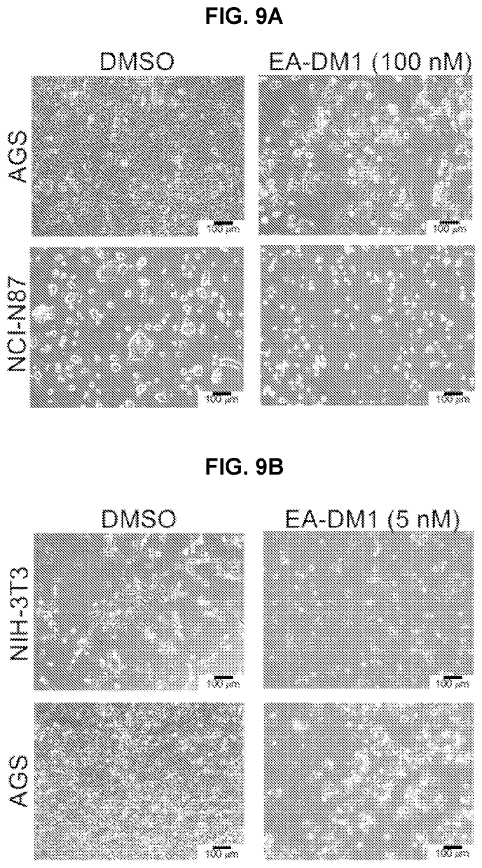

To evaluate whether EA-DM1 (cis-11-eicosenoic acid conjugated to mertansine (DM1)) could be internalized and release free DM1 toxin into the cytosol, two different gastric cancer cell lines, AGS and NCI-N87, were treated with EA-DM1 for 2 days (FIG. 9A-B). Gastric cancer cells died within 2 days after the EA-DM1 treatment at 100 nM concentration (FIG. 9A). Further, EA-DM1 effectively targeted gastric cancer cells at a lower concentration of 5 nM (FIG. 9B). In contrast, there were no noticeable changes in NIH-3T3 cells, a normal mouse fibroblast cell line, treated with 5 nM EA-DM1, indicating that the EA-DM1 does not target normal mouse cells (FIG. 9B). These results suggest that EA-DM1 intake can specifically target cancer cells which utilize fatty acid metabolism.

What is claimed:

1. A compound of formula (I): 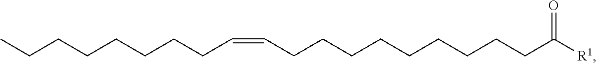
wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine.

2. The compound of claim 1, wherein the compound is: 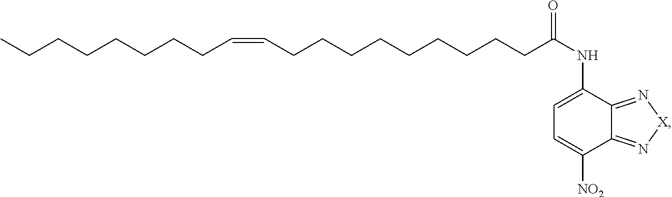
where X is O or Se.

3. A compound of formula (I): 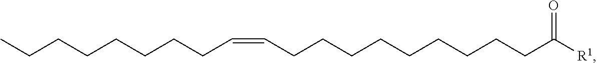
wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.

4. The compound of claim 3, wherein the compound is:

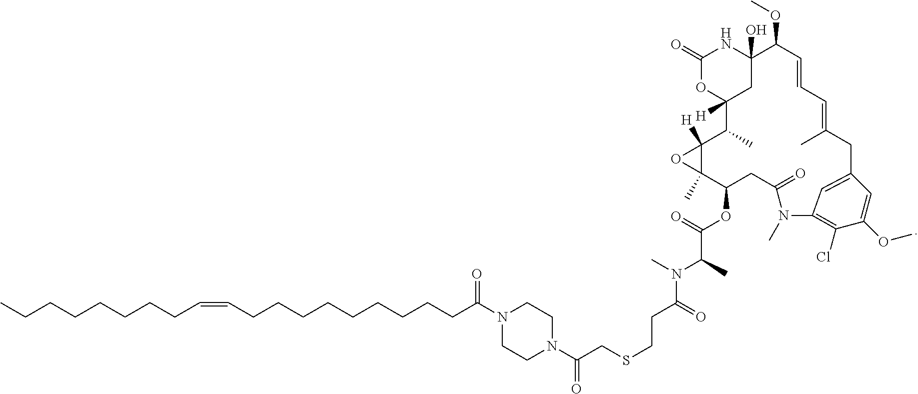 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

5. A method for detecting epithelial cell dysplasia, the method comprising:
contacting an epithelial cell with the compound of claim 1;
incubating for a period of time;
irradiating the epithelial cell with ultraviolet light, whereby a dysplastic epithelial cell will fluoresce; and
imaging the fluorescent dysplastic epithelial cell.

6. The method of claim 5, wherein imaging comprises positron emission tomography (PET) imaging, mass spectrometry imaging, immunofluorescence imaging, fluorescence molecular endoscopy, or fluorescence-guided intraluminal endoscopy.

7. The method of claim 5, wherein the epithelial cell comprises a gastric epithelial cell.

8. A research tool for detecting epithelial dysplasia, the research tool comprising a compound of formula (I): 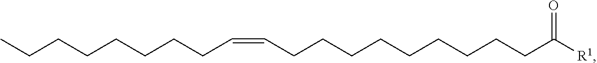
wherein R¹ is a fluorophore or a luminescent label selected from 7-nitrobenzo[c][1,2,5]oxadiazol-4-amine (nitrobenzofurazan), nitrobenzoselenadiazole, fluorescein, rhodamine, aminomethylcoumarin acetate (AMCA), calcein, or cyanine.

9. The research tool of claim 8, wherein the compound is: 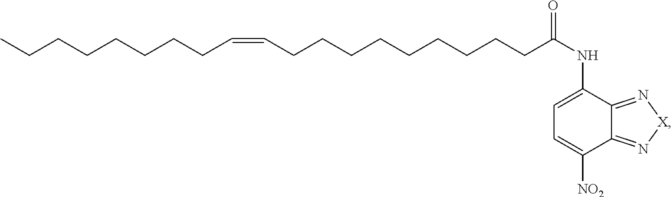
where X is O or Se.

10. A research tool comprising a compound of formula (I): 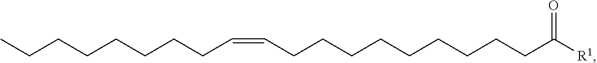
wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.

11. The research tool of claim 10, wherein the compound is:

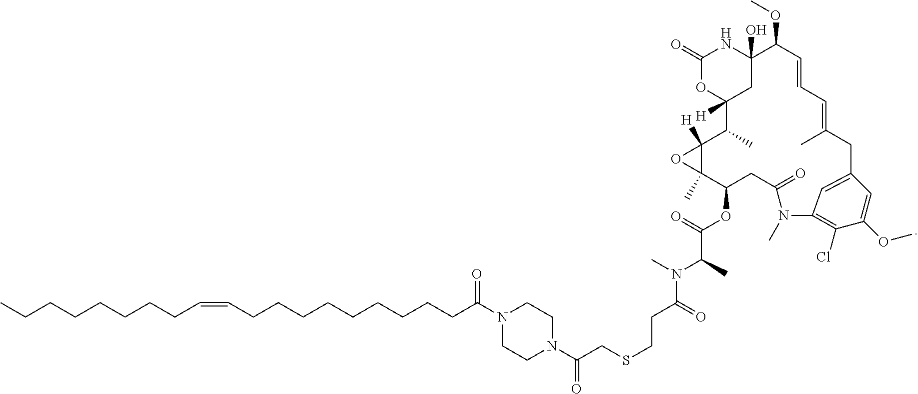 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

12. A method of inhibiting cancer or precancer in a cell or a subject in need thereof, the method comprising administering to the cell or the subject a therapeutically effective amount of a compound of formula (I): 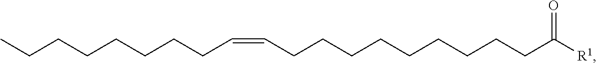
wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.

13. The method of claim 12, wherein the compound is:

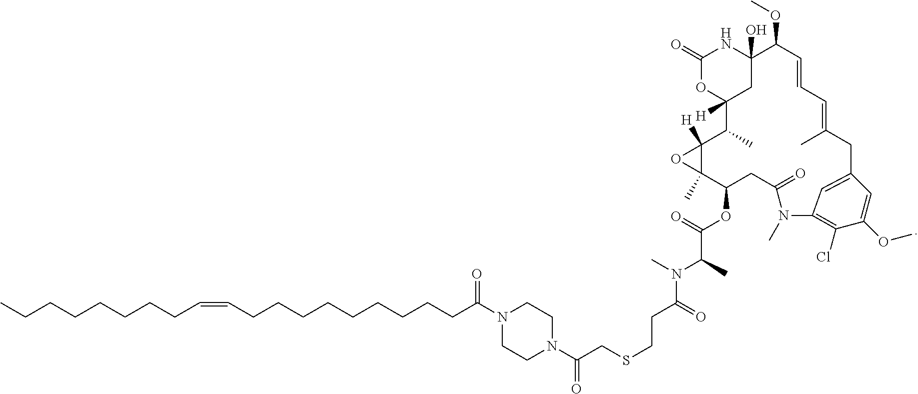 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

14. A method of treating a subject having cancer or precancer, the method comprising administering to the subject a therapeutically effective amount of a compound of formula (I): 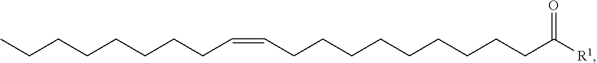
wherein R¹ is selected from mertansine (DM1), taxol, calicheamicin, monomethyl auristatin E (MMAE), deruxtecan, SN-38, Pseudomonas exotoxin, diphtheria toxin, or Yttrium-90.

15. The method of claim 14, wherein the compound is:

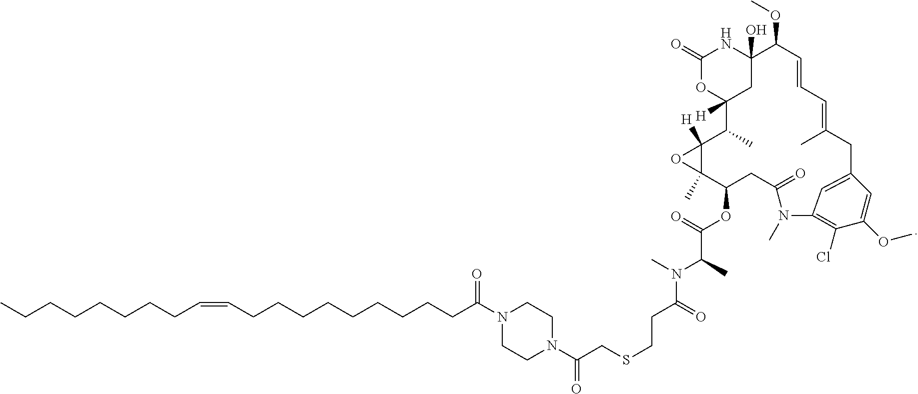 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]

16. The method of claim 14, wherein the subject has gastrointestinal cancer or precancer, or is suspected of developing gastrointestinal cancer or precancer.

17. A method for inhibiting the transition of a metaplastic cell to a dysplastic cell, the method comprising administering a therapeutically effective amount of 4-(2-chlorophenoxy)-N-(3-(methylcarbamoyl)phenyl)piperidine-1-carboxamide (A939572) to a cell or a subject in need thereof.

18. The method of claim 17, wherein the therapeutically effective amount of A939572 is from about 5 mg/kg to about 100 mg/kg.

19. The method of claim 17, wherein the therapeutically effective amount of A939572 is administered on a daily basis for about 1 day to about 6 months.

20. The method of claim 17, wherein the cell comprises a gastric epithelial cell.

21. The method of claim 17, wherein the subject has gastrointestinal cancer or precancer, or is suspected of developing gastrointestinal cancer or precancer.

22. A method for treating a metaplastic cell to reduce the incidence of carcinoma, the method comprising administering a therapeutically effective amount of 4-(2-chlorophenoxy)-N-(3-(methylcarbamoyl)phenyl)piperidine-1-carboxamide (A939572) to a cell or a subject in need thereof.

23. The method of claim 22, wherein the method inhibits the transition of the metaplastic cell to a dysplastic cell, thereby reducing the incidence of carcinoma in the cell or the subject in need thereof.

24. The method of claim 22, wherein the therapeutically effective amount of A939572 is from about 5 mg/kg to about 100 mg/kg.

25. The method of claim 22, wherein the therapeutically effective amount of A939572 is administered on a daily basis for about 1 day to about 6 months.

26. The method of claim 22, wherein the cell comprises a gastric epithelial cell.

27. The method of claim 22, wherein the subject has gastrointestinal cancer or precancer, or is suspected of developing gastrointestinal cancer or precancer.

28. A method of making a compound comprising cis-11-eicosenoic acid conjugated to 7-nitrobenzo[c][1,2,5]oxadiazol-4-amine (nitrobenzofurazan) or nitrobenzoselenadiazole: 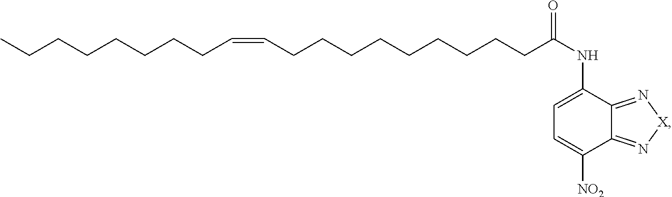
where X is O or Se;
the method comprising:
(a) combining cis-11-eicosenoic acid with dimethylformamide (DMF) solvent to create a solution;
(b) adding N,N-diisopropylethylamine (DIPEA) to the solution;
(c) adding 2-(1H-benzotriazole-1-yl)-1,1,3,3-tetramethyluronium hexafluorophosphate (HBTU) to the solution to create a reaction mixture;
(d) mixing the reaction mixture;
(e) adding nitrobenzofurazan or nitrobenzoselenadiazole to the reaction mixture to create a conjugate mixture;
(f) mixing the conjugate mixture; and
(g) one or more of diluting, washing, drying, or filtering the conjugate mixture to generate the compound.

29. The method of claim 28, wherein steps (a)-(e) are performed at about 0° C. and steps (f)-(g) are performed at room temperature.

30. The method of claim 28, wherein the conjugate mixture is diluted with ethyl acetate and washed with aqueous citric acid.

31. The method of claim 28, wherein the conjugate mixture is dried over MgSO₄.

32. The method of claim 28, further comprising purifying the compound using column chromatography.

33. A method of making a compound comprising cis-11-eicosenoic acid conjugated to mertansine: 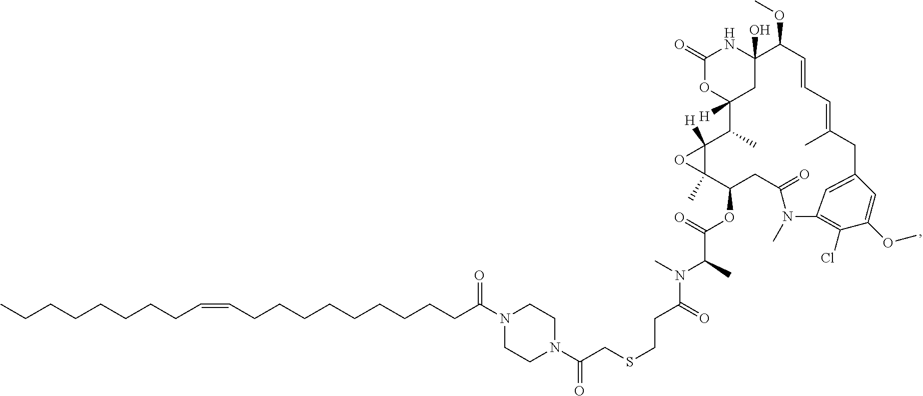 [SMILES: CCCCCCCC/C=C\CCCCCCCCCC(=O)N1CCN(C(=O)CSCCC(=O)N(C)[C@H](C)C(=O)O[C@@H]2CC(=O)N(C)c3cc(cc(OC)c3Cl)C/C(C)=C/C=C/[C@H](OC)[C@]3(O)C[C@@H](OC(=O)N3)[C@H](C)[C@H]3O[C@@]32C)CC1]
the method comprising:
(a) combining mertansine carboxylic acid with dimethylformamide (DMF) solvent to create a solution;
(b) adding hexafluorophosphate azabenzotriazole tetramethyl uranium (HATU) and N-methylmorpholine (NMM) to the solution to create a reaction mixture;
(c) mixing the reaction mixture;
(d) adding piperazine-conjugated cis-11-eicosenoic acid to the reaction mixture to create a conjugate mixture;
(e) mixing the conjugate mixture; and
(f) one or more of diluting, washing, drying, or filtering the conjugate mixture to generate the compound.
